(19) United States
(12) Patent Application Publication
(10) Pub. No.: US 2023/0149389 A1
(43) Pub. Date: May 18, 2023

(54) METHODS OF REDUCING MORTALITY IN SUBJECTS SUFFERING FROM AN UNDERLYING DISEASE OR CONDITION BY ADMINISTRATION OF METHYLNALTREXONE

(71) Applicant: Bausch Health Ireland Limited, Dublin (IE)

(72) Inventor: Neal Slatkin, Bridgewater, NJ (US)

(73) Assignee: Bausch Health Ireland Limited, Dublin (IE)

(21) Appl. No.: 17/965,394

(22) Filed: Oct. 13, 2022

Related U.S. Application Data
(63) Continuation of application No. PCT/EP2021/061453, filed on Apr. 30, 2021.
(60) Provisional application No. 63/113,062, filed on Nov. 12, 2020.
(60) Provisional application No. 63/019,301, filed on May 2, 2020.

(51) Int. Cl. A61K 31/485; A61P 35/00; A61P 43/00; A61K 9/00

(52) U.S. Cl. CPC A61K 31/485; A61P 35/00; A61P 43/00; A61K 9/0019

(57) ABSTRACT

The present disclosure is directed to the use of methylnaltrexone or a salt thereof or an ion pair thereof for reducing mortality in a subject suffering from an underlying disease or condition (e.g., advanced illness, cancer, a chronic non-cancer pain, postoperative ileus, or recovery from orthopedic surgery).

### CROSS REFERENCE TO RELATED APPLICATIONS

This application is a continuation of International Application No. PCT/EP2021/061453, filed on Apr. 30, 2021, which claims the benefit of U.S. Provisional Application No. 63/019,301, filed May 2, 2020, and U.S. Provisional Application No. 63/113,062, filed Nov. 12, 2020, the entirety of each of which are incorporated herein by reference.

### BACKGROUND

Evidence has emerged that people with cancer and noncancer pain who are treated with opioid analgesics may have an increased mortality risk beyond that attributable to opioid overdose alone. This finding has been exemplified in analyses of commercial, Medicare, and Medicaid claims databases, which suggest that patients using opioids for noncancer pain over extended periods have an elevated risk of all-cause mortality compared with the general population or with patients receiving other analgesics, even when deaths from accidental overdose are excluded. Preclinical and clinical studies have implied that activation of the μ-opioid receptor may influence clinical parameters of overall survival (OS) in patients with cancer and noncancer syndromes. In patients with newly diagnosed advanced cancer, higher μ-opioid receptor (MOR) expression and greater opioid requirements were associated with reductions in OS and shorter progression-free survival.

Accordingly, new therapies and formulations are desired in the field to reduce the incidence of all-cause mortality in patients treated with an opioid therapy.

### SUMMARY OF INVENTION

The invention described herein meets a present need in the field by reducing all-cause mortality risk in subjects in need thereof comprising administering an amount of one or more pharmaceutical compositions described herein that include methylnaltrexone (MNTX) or a salt or ion pair thereof. As further described herein, MNTX pharmaceutical compositions have been surprisingly found to significantly reduce the incidence of all-cause mortality in subjects treated with an opioid therapy. In certain embodiments, the opioid therapy may be an opioid analgesic therapy.

In an embodiment, the invention described herein includes a method for reducing mortality risk in a subject, wherein the subject may be receiving opioid therapy and/or suffering from an underlying disease or condition, comprising administering a pharmaceutical composition described herein to the subject. In some embodiments, the subject may be receiving opioid therapy. In some embodiments, the subject may be suffering from an underlying disease or condition. In some embodiments, the subject may be receiving opioid therapy and suffering from an underlying disease or condition. In some embodiments, the subject does not have opioid-induced bowel dysfunction (OIBD) or opioid induced constipation (OIC).

In an embodiment, the invention includes a method for reducing mortality risk in a subject, wherein the subject is receiving opioid therapy and is not suffering from an underlying disease or condition, comprising administering a pharmaceutical composition described herein to the subject.

In an embodiment, the invention includes a method for reducing mortality risk in a subject, wherein the subject is receiving opioid therapy and is suffering from, and/or has previously suffered from, opioid-induced bowel dysfunction (OIBD) or opioid induced constipation (OIC), comprising administering a pharmaceutical composition described herein to the subject.

In an embodiment, the invention includes a method for reducing mortality risk in a subject, wherein the subject is receiving opioid therapy but is not suffering from, and/or has not previously suffered from, opioid-induced bowel dysfunction (OIBD) or opioid induced constipation (OIC), comprising administering a pharmaceutical composition described herein to the subject.

In an embodiment, the invention includes a method for reducing mortality risk in a subject, wherein the method further comprises determining whether a subject has, and/or has previously had, an increased risk of death based on the presence of various risk factors, optionally, as listed under Table 3, and/or wherein the subject is a woman, is less than 60 years of age, has cancer, and/or has a chronic condition (i.e., an advanced illness, a cancer, and/or a chronic non-cancer pain), the method comprising administering a pharmaceutical composition described herein to the subject.

In an embodiment, the invention includes a method for reducing mortality risk in a subject, wherein the subject is receiving opioid therapy but is not suffering from, and/or has not previously suffered from, opioid-induced bowel dysfunction (OIBD) or opioid induced constipation (OIC), wherein the method further comprises determining whether a subject has, and/or has previously had, an increased risk of death based on the presence of various risk factors, optionally, as listed under Table 3, and/or wherein the subject is a woman, is less than 60 years of age, has cancer, and/or has a chronic condition (i.e., an advanced illness, a cancer, and/or a chronic non-cancer pain), the method comprising administering a pharmaceutical composition described herein to the subject.

In an embodiment, the invention includes a method of ameliorating opioid analgesic related mortality risk in a subject in need thereof, comprising administering a pharmaceutical composition described herein to the subject, wherein the subject is a woman, is less than 60 years of age, has cancer, and/or has a chronic condition (i.e., an advanced illness, a cancer, and/or a chronic non-cancer pain). In some embodiments, the subject does not have, and/or has not previously suffered from, opioid-induced bowel dysfunction (OIBD) or opioid induced constipation (OIC).

In an embodiment, the invention includes a method of ameliorating opioid analgesic related mortality risk in a subject in need thereof, comprising administering a pharmaceutical composition described herein to the subject, wherein the subject is a woman.

In an embodiment, the invention includes a method of ameliorating opioid analgesic related mortality risk in a subject in need thereof, comprising administering a pharmaceutical composition described herein to the subject, wherein the subject is less than 60 years of age.

In an embodiment, the invention includes a method of ameliorating opioid analgesic related mortality risk in a subject in need thereof, comprising administering a pharmaceutical composition described herein to the subject, wherein the subject has cancer.

In an embodiment, the invention includes a method of ameliorating opioid analgesic related mortality risk in a subject in need thereof, comprising administering a pharmaceutical composition described herein to the subject, wherein the subject does not have cancer.

In an embodiment, the invention includes a method of ameliorating opioid analgesic related mortality risk in a subject in need thereof, comprising administering a pharmaceutical composition described herein to the subject, wherein the subject has a chronic condition (i.e., an advanced illness, a cancer, and/or a chronic non-cancer pain).

In an embodiment, the invention includes a method of ameliorating opioid analgesic related mortality risk in a subject in need thereof, comprising administering a pharmaceutical composition described herein to the subject, wherein the subject does not have, and/or has not previously suffered from, opioid-induced bowel dysfunction (OIBD) or opioid induced constipation (OIC).

In an embodiment, the invention includes a method of ameliorating opioid analgesic related mortality risk in a subject in need thereof, comprising administering a pharmaceutical composition described herein to the subject, wherein the subject is receiving (or has received) opioid analgesic therapy.

In an embodiment, the invention includes a method for reducing mortality risk in a subject (as described herein) comprising administering one or more of the pharmaceutical compositions described herein.

In an embodiment, the invention includes a method for reducing mortality risk in a subject receiving opioid therapy (as described herein) comprising administering one or more of the pharmaceutical compositions described herein. In some embodiments, the subject is suffering from an underlying disease or condition. In some embodiments, the method for reducing mortality risk in a subject receiving opioid therapy comprises administering a therapeutically effective amount of one or more of the pharmaceutical compositions described herein. In some embodiments, the pharmaceutical composition may be one or more of a methylnaltrexone pharmaceutical composition for oral administration as described herein, a methylnaltrexone ion pair pharmaceutical composition as described herein, and a methylnaltrexone pharmaceutical composition for parenteral and subcutaneous administration as described herein. In some embodiments, the underlying disease or condition may be selected from the group consisting of an advanced illness, cancer, a chronic non-cancer pain, postoperative ileus, and recovery from orthopedic surgery, and combinations thereof.

In an embodiment, the invention includes a method for reducing mortality risk in a subject with a cancer and receiving opioid therapy (as described herein) comprising administering one or more of the pharmaceutical compositions described herein. In some embodiments, the method for reducing mortality risk in a subject receiving opioid therapy comprises administering a therapeutically effective amount of one or more of the pharmaceutical compositions described herein. In some embodiments, the pharmaceutical composition may be one or more of a methylnaltrexone pharmaceutical composition for oral administration as described herein, a methylnaltrexone ion pair pharmaceutical composition as described herein, and a methylnaltrexone pharmaceutical composition for parenteral and subcutaneous administration as described herein.

In an embodiment, the invention includes a method for reducing mortality risk in a subject less than 60 years of age receiving opioid therapy (as described herein) comprising administering one or more of the pharmaceutical compositions described herein. In some embodiments, the subject is suffering from an underlying disease or condition. In some embodiments, the method for reducing mortality risk in a subject receiving opioid therapy comprises administering a therapeutically effective amount of one or more of the pharmaceutical compositions described herein. In some embodiments, the pharmaceutical composition may be one or more of a methylnaltrexone pharmaceutical composition for oral administration as described herein, a methylnaltrexone ion pair pharmaceutical composition as described herein, and a methylnaltrexone pharmaceutical composition for parenteral and subcutaneous administration as described herein. In some embodiments, the underlying disease or condition may be selected from the group consisting of an advanced illness, cancer, a chronic non-cancer pain, postoperative ileus, and recovery from orthopedic surgery, and combinations thereof. In some embodiments, the subject is less than 50 years of age, or less than 40 years of age, or less than 30 years of age.

In an embodiment, the invention includes a method for reducing mortality risk in a subject with a chronic condition receiving opioid therapy (as described herein) comprising administering one or more of the pharmaceutical compositions described herein. In some embodiments, the method for reducing mortality risk in a subject receiving opioid therapy comprises administering a therapeutically effective amount of one or more of the pharmaceutical compositions described herein. In some embodiments, the pharmaceutical composition may be one or more of a methylnaltrexone pharmaceutical composition for oral administration as described herein, a methylnaltrexone ion pair pharmaceutical composition as described herein, and a methylnaltrexone pharmaceutical composition for parenteral and subcutaneous administration as described herein. In some embodiments, the chronic condition may be selected from the group consisting of an advanced illness, cancer, a chronic non-cancer pain, and combinations thereof.

In an embodiment, the invention includes a method for reducing mortality risk in a subject less than 60 years of age receiving opioid therapy (as described herein) comprising administering one or more of the pharmaceutical compositions described herein. In some embodiments, the subject is suffering from an underlying disease or condition. In some embodiments, the method for reducing mortality risk in a subject receiving opioid therapy comprises administering a therapeutically effective amount of one or more of the pharmaceutical compositions described herein. In some embodiments, the pharmaceutical composition may be one or more of a methylnaltrexone pharmaceutical composition for oral administration as described herein, a methylnaltrexone ion pair pharmaceutical composition as described herein, and a methylnaltrexone pharmaceutical composition for parenteral and subcutaneous administration as described herein. In some embodiments, the underlying disease or condition may be selected from the group consisting of an advanced illness, cancer, a chronic non-cancer pain, postoperative ileus, and recovery from orthopedic surgery, and combinations thereof. In some embodiments, the subject is less than 50 years of age, or less than 40 years of age, or less than 30 years of age.

In an embodiment, the invention includes a method for reducing mortality risk in a subject with a chronic condition receiving opioid therapy (as described herein) comprising administering one or more of the pharmaceutical compositions described herein. In some embodiments, the method for reducing mortality risk in a subject receiving opioid therapy comprises administering a therapeutically effective amount of one or more of the pharmaceutical compositions described herein. In some embodiments, the pharmaceutical composition may be one or more of a methylnaltrexone pharmaceutical composition for oral administration as described herein, a methylnaltrexone ion pair pharmaceutical composition as described herein, and a methylnaltrexone pharmaceutical composition for parenteral and subcutaneous administration as described herein. In some embodiments, the chronic condition may be selected from the group consisting of an advanced illness, cancer, a chronic non-cancer pain, and combinations thereof.

### BRIEF DESCRIPTION OF THE DRAWINGS

FIG. 1 depicts a Kaplan-Meier analysis and plot of all-cause mortality over time in the pooled methylnaltrexone and placebo groups from phase 2 to phase 4 randomized, double-blind, placebo-controlled studies. CI, confidence interval; HR, hazard ratio; MNTX, methylnaltrexone.

FIG. 2 depicts a comparison of mortality risk based on hazard ratios (95% CI) for the overall population and for each subgroup. *P<0.001; ^{†}P<0.01; ^{‡}P<0.05. CI, confidence interval.

FIG. 3 depicts a Kaplan-Meier plot of time to death by cancer and noncancer diagnosis. CI, confidence interval; HR, hazard ratio; MNTX, methylnaltrexone.

FIG. 4 depicts a Kaplan-Meier plot of time to death by age. CI, confidence interval; HR, hazard ratio; MNTX, methylnaltrexone.

FIG. 5 depicts a Kaplan-Meier plot of time to death by gender. CI, confidence interval; HR, hazard ratio; MNTX, methylnaltrexone.

FIG. 6 depicts a Kaplan-Meier analysis of mortality risk by diagnosis. CI, confidence interval; HR, hazard ratio; MNTX, methylnaltrexone.

### DETAILED DESCRIPTION

The invention described herein is based, at least in part, on the surprising discovery of a statistically and clinically significant reduction in all-cause mortality among patients receiving the μ-opioid receptor antagonist methylnaltrexone (MNTX) for treatment of opioid-induced bowel disorders (OIBD) compared with patients receiving placebo. More specifically, it has been identified that this mortality risk is stratified across certain patient subgroups, e.g., by underlying disease or condition, age, gender, and acute or chronic diagnosis. As discussed further herein, MNTX μ-opioid receptor antagonism may provide a protective benefit against the additional mortality risk associated with opioid treatment without interfering with analgesia in patients with cancer or chronic diagnoses.

Accordingly, in one aspect, the invention is directed to a method of reducing mortality in a subject suffering from an underlying disease or condition (e.g., an advanced illness, cancer, a chronic non-cancer pain, postoperative ileus, or recovery from orthopedic surgery), comprising administering to the subject an effective amount of a composition comprising methylnaltrexone (MNTX), or a salt thereof or an ion pair thereof.

In other aspects, the invention includes a pharmaceutical composition for reducing mortality in a subject suffering from an underlying disease or condition (e.g., an advanced illness, cancer, a chronic non-cancer pain, postoperative ileus, or recovery from orthopedic surgery).

### Definitions

Unless otherwise defined herein, scientific and technical terms used herein shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including,” as well as other forms of the term, such as “includes” and “included”, is not limiting.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

Furthermore, the transitional terms “comprising”, “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed invention. All embodiments described herein that encompass the invention can, in alternate embodiments, be more specifically defined by any of the transitional terms “comprising,” “consisting essentially of,” and “consisting of.”

The term “cancer” refers to a class of diseases characterized by abnormal cell proliferation. The term “abnormal cell proliferation” refers to abnormal, pathological, dysregulated and/or undesirable or inappropriate proliferation, division, growth or migration of cells that is not part of normal cell turnover, metabolism, growth or propagation, and generally is occurring more rapidly or to a significantly greater extent than typically occurs in a normally functioning cell of the same type and does not serve normal function. Abnormal cell proliferation and unwanted migration is manifest in disorders that are hyperproliferative in nature and include, but are not limited to, cancers, such as melanoma, lung cancer, breast cancer, pancreatic cancer, prostate cancer, colon cancer, ovarian cancer, head and neck cancer, leukemia, myeloma and/or solid tumor cancers. For example, the cancer can be one or more of a carcinoma, sarcoma, lymphoma, leukemia or blastoma. Solid tumor cancers, include, for example, adrenal cortical carcinoma, tumors of the bladder: squamous cell carcinoma, urothelial carcinomas; tumors of the bone (e.g., adamantinoma, aneurysmal bone cysts, chondroblastoma, chondroma, chondromyxoid fibroma, chondrosarcoma, fibrous dysplasia of the bone, giant cell tumor, osteochondroma, osteosarcoma); breast tumors (e.g., secretory ductal carcinoma, chordoma); colon tumors (e.g, colorectal adenocarcinoma); eye tumors (e.g., posterior uveal melanoma, fibrogenesis imperfecta ossium, head and neck squamous cell carcinoma); kidney tumors (e.g., chromophobe renal cell carcinoma, clear cell renal cell carcinoma, nephroblastoma (Wilms tumor), papillary renal cell carcinoma, primary renal ASPSCR1-TFE3 tumor, renal cell carcinoma); liver tumors (e.g., hepatoblastoma, hepatocellular carcinoma); lung tumors (e.g., non-small cell carcinoma, small cell cancer; malignant melanoma of soft parts); nervous system tumors (e.g., medulloblastoma, meningioma, neuroblastoma, astrocytic tumors, ependymomas, peripheral nerve sheath tumors, phaeochromocytoma); ovarian tumors (e.g., epithelial tumors, germ cell tumors, sex cord-stromal tumors, pericytoma; pituitary adenomas); rhabdoid tumor; skin tumors (e.g., cutaneous benign fibrous histiocytomas); smooth muscle tumors (e.g., intravenous leiomyomatosis); soft tissue tumors (e.g., liposarcoma, myxoid liposarcoma, low grade fibromyxoid sarcoma, leiomyosarcoma, alveolar soft part sarcoma, angiomatoid fibrous histiocytoma (AFH), clear cell sarcoma, desmoplastic small round cell tumor, elastofibroma, Ewing's tumors, extraskeletal myxoid chondrosarcoma, inflammatory myofibroblastic tumor, lipoblastoma, lipoma/benign lipomatous tumors, liposarcoma/malignant lipomatous tumors, malignant myoepithelioma, rhabdomyosarcoma, synovial sarcoma, squamous cell cancer); tumors of the testis (e.g., germ cell tumors, spermatocyte seminoma); thyroid tumors (e.g., anaplastic (undifferentiated) carcinoma, oncocytic tumors, papillary carcinoma); and uterine tumors (e.g., carcinoma of the cervix, endometrial carcinoma, leiomyoma). In some embodiments, the tumor can include one or more of a breast, liver, breast, head and neck, liver, esophageal, stomach, small intestine, colon, rectal, anal, skin, glandular, circulatory, prostate, pancreas, hematopoietic, bone marrow, bone, cartilage, fat, nerve, or lymph tumor.

As used herein, an “effective amount” of a composition of methylnaltrexone, or a salt or ion pair thereof, refers to the level required to reduce mortality in a subject as described herein. In some embodiments, an “effective amount” is at least a minimal amount of methylnaltrexone, or a salt or ion pair thereof, which is sufficient for reducing mortality in a subject receiving opioid therapy. In some embodiments, an “effective amount” is at least a minimal amount of methylnaltrexone, or a salt or ion pair thereof, which is sufficient for reducing mortality in a subject suffering from an underlying disease or condition. An effective amount of a composition of methylnaltrexone, or salt or ion pair thereof, may vary according to factors such as the disease state, age, and weight of the subject, and the ability of a composition of methylnaltrexone, or a salt thereof, or an ion pair thereof to elicit a desired response in the subject. Dosage regimens may be adjusted to provide the optimum therapeutic response. An effective amount is also one in which any toxic or detrimental effects (e.g., side effects) of a composition of methylnaltrexone, or a salt or ion pair thereof, are outweighed by the therapeutically beneficial effects.

The terms “treat” or “treating,” as used herein, refer to partially or completely alleviating, inhibiting, delaying onset of, reducing the incidence of, mortality in a subject, e.g., using a pharmaceutical composition or method described herein.

The expression “unit dosage form” as used herein refers to a physically discrete unit of a composition or formulation of methylnaltrexone, or a salt thereof, or an ion pair thereof, appropriate for the subject to be treated. The specific effective dose level for any particular subject will depend upon a variety of factors including the severity of the underlying disease or condition; nature and activity of the composition; specific formulation employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

The term “subject”, as used herein, means a mammal and includes human and animal subjects, such as domesticated animals (e.g., horses, dogs, cats, etc.) and experimental animals (e.g., mice, rats, dogs, chimpanzees, apes, etc.). In a particular embodiment, the subject is a human.

The terms “suffer” or “suffering,” as used herein, refer to one or more conditions that a subject has been diagnosed with, or is suspected to have, in particular, an advanced illness, cancer, a chronic non-cancer pain, postoperative ileus, or recovery from orthopedic surgery.

The term “amphiphilic,” as used herein, to describe a molecule refers to the molecule's dual hydrophobic and hydrophilic properties. Typically, amphiphilic molecules have a polar, water soluble group (e.g., a phosphate, carboxylic acid, sulfate) attached to a nonpolar, water-insoluble group (e.g., a hydrocarbon). The term amphiphilic is synonymous with amphipathic. Examples of amphiphilic molecules include sodium dodecyl (lauryl) sulfate, fatty acids, phospholipids, and bile acids. Amphiphilic molecules may be uncharged, cationic, or anionic.

As used herein, the term “lipophilicity” refers to a compound's ability to associate with or dissolve in a fat, lipid, oil, or non-polar solvent. Lipophilicity and hydrophobicity may be used to describe the same tendency of a molecule to dissolve in fats, oils, lipids, and non-polar solvents.

As used herein, reducing or decreasing “mortality risk” or “all-cause mortality risk” refers to reducing or decreasing the likelihood of mortality (i.e., death) for a subject(s) and treated with a pharmaceutical composition described herein as compared to a similarly situated subject(s) that is not treated with a pharmaceutical composition described herein. For example, similarly situated subjects may be those subjects who (a) have cancer; (b) are women; (c) are less than 60 years of age; and/or (d) have a chronic condition (i.e., an advanced illness, a cancer, or chronic non-cancer pain). In a particular embodiment, the subject is a subject receiving opioid therapy and assessment of reduction of mortality risk or all-cause mortality is as compared to a similarly situated subject receiving opioid therapy.

The terms “administration” or “administering” include routes of introducing a composition(s) of methylnaltrexone, to a subject to perform their intended function. As used herein, administration of composition(s) of methylnaltrexone, or a salt thereof, or an ion pair thereof are intended to include injection (depot injection, subcutaneous, intravenous, parenterally, intraperitoneally, intrathecal), oral, subcutaneous, rectal, and transdermal. The pharmaceutical preparations may be given by forms suitable for each administration route. For example, for oral administration these preparations can be administered in tablet, liquid, or capsule form. Alternatively, these preparations can be administration by injection or infusion; topical administration, by lotion or ointment; and rectal by suppositories. Oral or subcutaneous administration is preferred. The injection can be bolus or can be continuous infusion. Depending on the route of administration, a composition(s) of methylnaltrexone, or a salt thereof, or an ion pair thereof, can be coated with or disposed in a selected material to protect it from natural conditions that may detrimentally affect its ability to perform its intended function. A composition(s) of methylnaltrexone, or a salt thereof, or an ion pair thereof, can be administered alone, or in conjunction with either another agent or agents as described herein or with a pharmaceutically acceptable carrier, or both.

In certain embodiments, the subject is administered the pharmaceutical composition as described herein at least once a day. In certain embodiments, the subject is administered the pharmaceutical composition as described herein at least twice a day. In certain embodiments, the subject is administered the pharmaceutical composition as described herein at least three times a day. In other embodiments, the subject is administered the pharmaceutical composition up to once a day. In other embodiments, the subject is administered the pharmaceutical composition up to twice a day. In other embodiments, the subject is administered the pharmaceutical composition up to three times a day. In certain embodiments, the subject is administered the pharmaceutical composition not more than once a day. In certain embodiments, the subject is administered the pharmaceutical composition not more than twice a day. In certain embodiments, the subject is administered the pharmaceutical composition not more than three times a day. In certain embodiments, the subject is administered the pharmaceutical composition as needed. In certain embodiments, the subject is administered the pharmaceutical composition as needed, but not more than once a day. In certain embodiments, the subject is administered the pharmaceutical composition as needed, but not more than twice a day. In certain embodiments, the subject is administered the pharmaceutical composition as needed, but not more than three times a day. In certain embodiments, the subject is administered the pharmaceutical composition at least once every other day. In certain embodiments, the subject is administered the pharmaceutical composition as described herein at least twice every other day. In certain embodiments, the subject is administered the pharmaceutical composition as described herein at least three times every other day.

Administration “in combination with” one or more further therapeutic agents include simultaneous (concurrent) and consecutive administration in any order.

As will be readily apparent to one skilled in the art, the useful in vivo dosage to be administered and the particular mode of administration will vary depending upon the age, weight and mammalian species treated, the particular compounds employed, and the specific use for which these compounds are employed. Typically, human clinical applications of products are commenced at lower dosage levels, with dosage level being increased until the desired effect is achieved.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN, polyethylene glycol (PEG).

### Methylnaltrexone Pharmaceutical Compositions

Pharmaceutical compositions and methods described herein include methylnaltrexone or a salt thereof or an ion pair thereof in a therapeutically effective amount. In some embodiments, the pharmaceutical composition referred to herein may be a methylnaltrexone pharmaceutical composition for oral administration as described herein, a methylnaltrexone ion pair pharmaceutical composition as described herein, or a methylnaltrexone pharmaceutical composition for parenteral and subcutaneous administration as described herein.

In some embodiments, the pharmaceutical compositions and methods described herein may include methylnaltrexone or a salt thereof or an ion pair thereof in an amount of about 1 to about 1000 mg of methylnaltrexone or a salt thereof or an ion pair thereof. In some embodiments, the pharmaceutical compositions and methods described may include methylnaltrexone or a salt thereof or an ion pair thereof in an amount of about 0.5 mg to about 500 mg, or about 1 mg to about 100 mg, or about 0.5 mg to about 200 mg, or about 50 mg to about 500 mg, or about 50 mg to about 900 mg, or about 75 mg to about 850 mg, or about 100 mg to about 850 mg, or about 150 mg to about 850 mg, or about 200 mg to about 800 mg, or about 200 mg to about 700 mg, or about 150 mg to about 450 mg, or about 10 mg to about 600 mg, or about 75 mg to about 900 mg.

In some embodiments, the pharmaceutical composition and methods described herein may include methylnaltrexone or a salt thereof or an ion pair thereof in an amount of at least about 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 mg.

In some embodiments, the pharmaceutical composition and methods described herein may include methylnaltrexone or a salt thereof or an ion pair thereof in an amount of at most about 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 mg.

In some embodiments, the pharmaceutical composition and methods described herein may include methylnaltrexone or a salt thereof or an ion pair thereof in an amount of about 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 mg.

In some embodiments, the pharmaceutical composition and methods described herein may include methylnaltrexone or a salt thereof or an ion pair thereof in an amount to provide a dosage to the subject of about 0.01 mg/kg to about 100 mg/kg, about 0.01 mg/kg to about 80 mg/kg, about 1 mg/kg to about 100 mg/kg, or about 5 mg/kg to about 75 mg/kg, or about 25 mg/kg to about 75 mg/kg.

In some embodiments, the pharmaceutical composition and methods described herein may include methylnaltrexone or a salt thereof or an ion pair thereof in an amount to provide a dosage to the subject of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg.

In some embodiments, the pharmaceutical composition and methods described herein may include methylnaltrexone or a salt thereof or an ion pair thereof in an amount to provide a dosage to the subject of at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg.

In some embodiments, the pharmaceutical composition and methods described herein may include methylnaltrexone or a salt thereof or an ion pair thereof in an amount to provide a dosage to the subject of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg.

In some embodiments, the pharmaceutical composition includes about 1% to about 75%, about 10% to about 60%, about 15% to about 50%, or about 20% to about 40% by weight, of methylnaltrexone or a salt thereof or an ion pair thereof. In some embodiments, the pharmaceutical composition includes at least 1%, or at least 2%, or at least 3%, or at least 4%, or at least 5%, or at least 6%, or at least 7%, or at least 8%, or at least 9%, or at least 10%, or at least 11%, or at least 12%, or at least 13%, or at least 14%, or at least 15%, or at least 16%, or at least 17%, or at least 18%, or at least 19%, or at least 20%, or at least 21%, or at least 22%, or at least 23%, or at least 24%, or at least 25%, or at least 26%, or at least 27%, or at least 28%, or at least 29%, or at least 30%, or at least 31%, or at least 32%, or at least 33%, or at least 34%, or at least, 35%, or at least 36%, or at least 37%, or at least 38%, or at least 39%, or at least 40%, or at least 41%, or at least 42%, or at least 43%, or at least 44%, or at least 45%, or at least 46%, or at least 47%, or at least 48%, or at least 49%, or at least 50%, or at least 51%, or at least 52%, or at least 53%, or at least 54%, or at least 55%, or at least 56%, or at least 57%, or at least 58%, or at least 59%, or at least 60% by weight, of methylnaltrexone or a salt thereof or an ion pair thereof. In some embodiments, the pharmaceutical composition includes at most 1%, or at most 2%, or at most 3%, or at most 4%, or at most 5%, or at most 6%, or at most 7%, or at most 8%, or at most 9%, or at most 10%, or at most 11%, or at most 12%, or at most 13%, or at most 14%, or at most 15%, or at most 16%, or at most 17%, or at most 18%, or at most 19%, or at most 20%, or at most 21%, or at most 22%, or at most 23%, or at most 24%, or at most 25%, or at most 26%, or at most 27%, or at most 28%, or at most 29%, or at most 30%, or at most 31%, or at most 32%, or at most 33%, or at most 34%, or at most, 35%, or at most 36%, or at most 37%, or at most 38%, or at most 39%, or at most 40%, or at most 41%, or at most 42%, or at most 43%, or at most 44%, or at most 45%, or at most 46%, or at most 47%, or at most 48%, or at most 49%, or at most 50%, or at most 51%, or at most 52%, or at most 53%, or at most 54%, or at most 55%, or at most 56%, or at most 57%, or at most 58%, or at most 59%, or at most 60% by weight, of methylnaltrexone or a salt thereof or an ion pair thereof. In some embodiments, the pharmaceutical composition includes about 1%, or about 2%, or about 3%, or about 4%, or about 5%, or about 6%, or about 7%, or about 8%, or about 9%, or about 10%, or about 11%, or about 12%, or about 13%, or about 14%, or about 15%, or about 16%, or about 17%, or about 18%, or about 19%, or about 20%, or about 21%, or about 22%, or about 23%, or about 24%, or about 25%, or about 26%, or about 27%, or about 28%, or about 29%, or about 30%, or about 31%, or about 32%, or about 33%, or about 34%, or about 35%, or about 36%, or about 37%, or about 38%, or about 39%, or about 40%, or about 41%, or about 42%, or about 43%, or about 44%, or about 45%, or about 46%, or about 47%, or about 48%, or about 49%, or about 50%, or about 51%, or about 52%, or about 53%, or about 54%, or about 55%, or about 56%, or about 57%, or about 58%, or about 59%, or about 60% by weight, of methylnaltrexone or a salt thereof or an ion pair thereof.

In various embodiments, the pharmaceutical composition may include a therapeutically effective amount of methylnaltrexone or a salt thereof or an ion pair thereof and a pharmaceutically acceptable carrier. In some embodiments, the pharmaceutical composition may include a methylnaltrexone or a salt thereof or an ion pair thereof in an amount or dosage described herein and a pharmaceutically acceptable carrier. In some embodiments, the pharmaceutically acceptable carrier may include one or more of the carriers or excipients described herein.

In some embodiments, the invention includes a pharmaceutical composition comprising methylnaltrexone or a salt thereof or an ion pair thereof, wherein the methylnaltrexone or salt thereof or an ion pair thereof is provided in an amount of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90% by weight of the pharmaceutical composition. In some embodiments, the invention includes a pharmaceutical composition comprising methylnaltrexone or a salt thereof or an ion pair thereof, wherein the methylnaltrexone or salt thereof is provided in an amount of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90% by weight of the pharmaceutical composition. In some embodiments, the invention includes a pharmaceutical composition comprising methylnaltrexone or a salt thereof or an ion pair thereof, wherein the methylnaltrexone or salt thereof, or an ion pair thereof, is provided in an amount of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90% by weight of the pharmaceutical composition.

In some embodiments, pharmaceutical compositions described herein may be suitable for oral administration and can be presented as discrete dosage forms, such as capsules, sachets, tablets, liquids, or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the pharmaceutical compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

In some embodiments, the pharmaceutical compositions described herein may encompasses anhydrous pharmaceutical compositions and dosage forms since water can facilitate the degradation of some compounds. For example, water may be added in the pharmaceutical arts as a means of simulating long-term storage in order to determine characteristics such as shelf-life or the stability of pharmaceutical compositions over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, pharmaceutical compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

In some embodiments of the pharmaceutical compositions described herein, methylnaltrexone can be combined in an intimate admixture with a pharmaceutical carrier according to conventional pharmaceutical compounding techniques. The carrier can take a wide variety of forms depending on the form of preparation desired for administration. In preparing pharmaceutical compositions for an oral dosage form, any of the usual pharmaceutical media can be employed as carriers, such as, for example, water, glycols (e.g., propylene glycol), oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers may include powders, capsules, and tablets, with the solid oral preparations. If desired, tablets can be coated by standard aqueous or nonaqueous techniques.

In some embodiments, the pharmaceutical compositions described herein may include on or more binders. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pregelatinized starch, hydroxypropyl methyl cellulose, microcrystalline cellulose, and mixtures thereof.

In some embodiments, the pharmaceutical compositions described herein may include one or more fillers. Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

In some embodiments, the pharmaceutical compositions described herein may include one or more disintegrants. Disintegrants may be used in the pharmaceutical compositions of the invention to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the active agents disclosed herein. The amount of disintegrant used may vary based upon the type of pharmaceutical composition and mode of administration, and may be readily discernible to those of ordinary skill in the art. About 0.5 to about 15 weight percent of disintegrant, or about 1 to about 5 weight percent of disintegrant, may be used in the pharmaceutical compositions. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

In some embodiments, the pharmaceutical compositions described herein may include one or more lubricants. Lubricants which can be used to form pharmaceutical compositions and dosage forms of the invention include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl aureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

In some embodiments, the pharmaceutical compositions described herein may be in tablet unit dosage form. The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Pharmaceutical compositions for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil. For example, a tablet coating may be an Opadry coating (e.g., Opadry II).

In some embodiments, the pharmaceutical compositions described herein may include one or more surfactants. Surfactants which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

In some embodiments, the pharmaceutical compositions may include a solubilizer to ensure good solubilization and/or dissolution of the compound described herein and to minimize precipitation of the compound described herein. A solubilizer may also be added to increase the solubility of the hydrophilic drug and/or other components, such as surfactants, or to maintain the pharmaceutical composition as a stable or homogeneous solution or dispersion. Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, E-caprolactam, N-alkylpyrrolidone, N-hydroxyalkylpyrrolidone, N-alkylpiperidone, N-alkyl caprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, caprolactone and isomers thereof, 8-valerolactone and isomers thereof, β-butyrolactone and isomers thereof and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but are not limited to, triacetin, triethylcitrate, ethyl oleate, ethyl caprylate, dimethylacetamide, N-methylpyrrolidone, N-hydroxyethylpyrrolidone, polyvinylpyrroli done, hydroxypropylmethyl cellulose, hydroxypropyl cyclodextrins, ethanol, polyethylene glycol 200-100, glycofurol, transcutol, propylene glycol, and dimethyl isosorbide.

In some embodiments, the pharmaceutical compositions described herein can further include one or more pharmaceutically acceptable additives. Such additives include, without limitation, detackifiers, antifoaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

Methylnaltrexone Pharmaceutical Compositions for Oral Administration

In some embodiments, methylnaltrexone provided in the pharmaceutical composition described herein may include methylnaltrexone and an amphiphilic pharmaceutically acceptable excipient. For example, the methylnaltrexone may be a salt of methylnaltrexone of the formula:

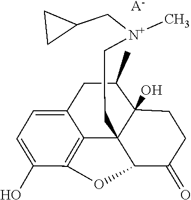

wherein methylnaltrexone is the cation of the salt, and A⁻ is an anion of an amphiphilic pharmaceutically acceptable excipient, as described in U.S. Pat. No. 8,524,276, the entire contents of which are hereby incorporated by reference herein. In certain embodiments, the methylnaltrexone is (R)—N-methylnaltrexone, a peripherally acting μ opioid receptor antagonist, as shown in the formula above. It will be understood that the (R)—N-methylnaltrexone cation and the anion of the amphiphilic pharmaceutically acceptable excipient may exist in the composition as an ion pair or may exist as separate salts paired with other counter ions. For example, methylnaltrexone included in the pharmaceutical compositions and methods of the invention described herein may be provided as (R)—N-methylnaltrexone bromide and sodium lauryl sulfate.

As set forth herein, pharmaceutical compositions and methods of the invention may include an amphiphilic pharmaceutically acceptable excipient or an anion thereof (A⁻). The amphiphilic pharmaceutically acceptable excipient increases the lipophilicity of the composition thereby allowing for increased transport through the unstirred diffusion layer in the GI tract, resulting in increased permeation through biological membranes. In certain embodiments, the excipient increases the lipophilicity of a methylnaltrexone.

In certain embodiments, the amphiphilic pharmaceutically acceptable excipient may include a sulfate, sulfonate, nitrate, nitrite, phosphate, or phosphonate moiety. In one embodiment, the pharmaceutically acceptable excipient comprises an (—OSO₃⁻) group. In certain embodiments, the anion is butyl sulfate, pentyl sulfate, hexyl sulfate, heptyl sulfate, octyl sulfate, nonyl sulfate, decyl sulfate, undecyl sulfate, dodecyl sulfate, tridecyl sulphate, tetradecyl sulfate, pentadecyl sulfate, hexadecyl sulfate, heptadecyl sulfate, octadecyl sulfate, eicosyl sulfate, docosyl sulfate, tetracosyl sulfate, hexacosyl sulfate, octacosyl sulfate, and triacontyl sulphate.

In certain embodiments, A⁻ is the anion of a Brønsted acid. Exemplary Brønsted acids include hydrogen halides, carboxylic acids, sulfonic acids, sulfuric acid, and phosphoric acid. In certain embodiments, A⁻ is chloride, bromide, iodide, fluoride, sulfate, bisulfate, tartrate, nitrate, citrate, bitartrate, carbonate, phosphate, malate, maleate, fumarate sulfonate, methylsulfonate, formate, carboxylate, sulfate, methylsulfate or succinate salt. In certain embodiments, A⁻ is trifluoroacetate or bromide.

In certain embodiments, the methylnaltrexone of the pharmaceutical compositions and methods described herein may have multiple anions (e.g., bromide and dodecyl (lauryl) sulfate) associating therewith.

In certain embodiments, A⁻ is bromide, such that methylnaltrexone may be (R)—N-methylnaltrexone bromide. (R)—N-methylnaltrexone bromide, which is also known as “MNTX” and is described in U.S. Pat. No. 8,343,992, which is incorporated herein by reference. The chemical name for (R)—N-methylnaltrexone bromide is (R)—N-(cyclopropylmethyl) noroxymorphone methobromide. (R)—N-methylnaltrexone bromide has the molecular formula C₂₁H₂₆NO₄Br and a molecular weight of 436.36 g/mol. (R)—N-methylnaltrexone bromide has the following structure:

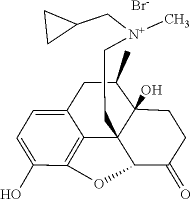 [SMILES: [Br-] ; C[C@@H]1Cc2ccc(O)c3c2[C@@]2(CCC[NH+](C)CC4CC4)[C@@H](O3)C(=O)CC[C@@]12O]

(R)—N-methylnaltrexone bromide
   where the compound is in the (R) configuration with respect to the quaternary nitrogen. In certain embodiments presented herein, at least about 99.6%, 99.7%, 99.8%, 99.85%, 99.9%, or 99.95% of the compound is in the (R) configuration with respect to nitrogen. Methods for determining the amount of (R)—N-methylnaltrexone bromide, present in a sample as compared to the amount of (S)—N-methylnaltrexone bromide present in that same sample, are described in U.S. Pat. No. 8,343,992, which is incorporated herein by reference. In other embodiments, the methylnaltrexone contains 0.15%, 0.10%, or less (S)—N-methylnaltrexone bromide.

In certain embodiments, A⁻ is an acidic amphiphilic pharmaceutically acceptable excipient. In certain embodiments, the pharmaceutically acceptable excipient has a pKₐ of about 3 or less. In certain embodiments, the pharmaceutically acceptable excipient has a pKₐ of about 2 or less. In certain embodiments, the pharmaceutically acceptable excipient has a pKₐ between about 1 and about 2. In certain embodiments, the pharmaceutically acceptable excipient has a pKₐ of about 1 or less.

In some embodiments, methylnaltrexone for use in pharmaceutical compositions and methods described herein may be provided in any of a variety of forms. For example, forms of methylnaltrexone suitable for use herein include pharmaceutically acceptable salts, prodrugs, polymorphs (i.e., crystal forms), co-crystals, hydrates, solvates, and the like. In some embodiments, the form of methylnaltrexone used should allow for association with the amphiphilic pharmaceutically acceptable excipient, for example, through ion pairing either in the pharmaceutical composition or in situ.

In some embodiments, pharmaceutical compositions and methods described herein that include oral delivery of methylnaltrexone may include an amphiphilic pharmaceutically acceptable excipient as described above, and a disintegrant, and further, optionally, comprise one or more other components, such as, for example, binders, carriers, chelating agents, antioxidants, fillers, lubricants, wetting agents, or combinations thereof, as set forth in U.S. Pat. No. 9,314,461, the entire contents of which are incorporated herein by reference.

In some embodiments, the pharmaceutical compositions and methods described herein includes a combination of methylnaltrexone bromide and sodium dodecyl (lauryl) sulfate (also known as SDS or SLS). In certain embodiments, the pharmaceutical compositions and methods described herein may include delivery of sodium bicarbonate as a disintegrant. Additional excipients, as set forth above, may be incorporated, including, but not limited to, at least one of microcrystalline cellulose, crospovidone, polysorbate 80, edetate calcium disodium dehydrate, silicified microcrystalline cellulose, talc, colloidal silicon dioxide and magnesium stearate. In one embodiment, the pharmaceutical compositions and methods described herein may include delivery of methylnaltrexone bromide, sodium lauryl sulfate, sodium bicarbonate, microcrystalline cellulose, crospovidone, polysorbate 80, edetate calcium disodium dehydrate, silicified microcrystalline cellulose, talc, colloidal silicon dioxide and magnesium stearate.

In some embodiments, methylnaltrexone is provided in the methods described herein as oral RELISTOR® tablets, an FDA approved tablet product for oral administration. In some embodiments, methylnaltrexone is provided in the methods described herein as one or more oral RELISTOR® tablets (150 mg).

### Methylnaltrexone Ion Pair Pharmaceutical Compositions for Oral Administration

In some embodiments, methylnaltrexone is provided in the methods described herein as a liquid oral dosage form, e.g., as described in International Application No. PCT/EP2020/062794 (published as International Publication No. WO 2020/225395), the entire contents of which are hereby incorporated by reference herein.

Methylnaltrexone is a quaternary amine and, as such, has a positive charge. This charge results in slower absorption rates (as compared to neutral molecules) across membranes. The existing RELISTOR® tablet, as described, for example, in U.S. Pat. No. 9,314,461, combines methylnaltrexone bromide with sodium lauryl sulfate, and relies on in situ formation of the neutral methylnaltrexone and lauryl sulfate ion pair to enhance absorption. Liquid oral dosage forms, e.g., as described in International Application No. PCT/EP2020/062794, however, are predicated, at least in part, on the finding that the formulation and administration of a pre-existing ion pair of methylnaltrexone and either docusate or lauryl sulfate, can serve to enhance the absorption rate. Because methylnaltrexone has been reported to have a high first-pass metabolism, an increase in the rate of absorption can serve to saturate the metabolic pathway, thereby further enhancing the amount of methylnaltrexone absorbed by the body.

In some embodiments, a pharmaceutical compositions in a liquid oral dosage form comprises: (a) an ion pair having the formula:

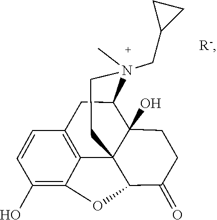

wherein R⁻ may be an anion selected to provide the ion pair; and (b) one or more of an oil, surfactant, or a cosolvent. In some embodiments, R⁻ may be an anion selected from the group consisting of lauryl sulfate and docusate. In particular embodiments, the liquid oral dosage form includes an oil and a surfactant. In some embodiments, the liquid oral dosage form includes a surfactant and a cosolvent.

In particular embodiments, the pharmaceutical compositions of the invention are formulated and administered as a capsule, e.g., soft gel capsule, hard gel capsule, and enteric gel capsule. In a particular embodiment, the pharmaceutical compositions are formulated as a soft gel capsule.

However, the compositions may alternatively be formulated as tablets. In some embodiments, solid granules can be produced by melt granulation. In other embodiments, waxy powders can be produced by solvent evaporation. In further embodiments, solid granules and/or powders can be produced by spray drying. Such granules and powders can be compressed into tablets in accordance with the inventive subject matter. In some embodiments, lipids are adsorbed onto a solid carrier, such as silicon dioxide, calcium silicate, and/or magnesium aluminometasilicate, which is compressed to make tablets.

Ion pairs, generally, are salts that include a hydrophilic active agent (cation or anion) and a more lipophilic pharmaceutically acceptable counterion. See e.g., Krisztina Tkács-Novák & György Száz, Ion-Pair Partition of Quaternary Ammonium Drugs: The Influence of Counter Ions of Different Lipophilicity, Size, and Flexibility, 16(10) PHARMACEUTICAL RESEARCH 1633-38 (1999). Ion pairs may have a greater hydrophobicity than the active agent as measured by a partition coefficient, e.g.,

$$log ( {P}_{\frac{o ctanol}{water}} ) .$$

Increasing the lipophilicity of methylnaltrexone through the pre-formation of ion pairs with lauryl sulfate and/or docusate, as described herein, can improve the ability of methylnaltrexone to penetrate membrane barriers and thereby enhance bioavailability and/or efficacy of methylnaltrexone oral formulations.

In certain embodiments, where the pharmaceutical composition includes an ion pair of methylnaltrexone and lauryl sulfate, the ion pair is present at about 1% to about 50%, about 5% to about 45%, about 10% to about 40%, about 10% to about 35%, about 10% to about 30%, or about 15% to about 25% by weight of the composition. For example, the methylnaltrexone and lauryl sulfate ion pair may be present in an amount of at least about 10%, 11%, 12%, 13%, 14%, 15%, 16%, 17%, 18%, 19%, 20%, 21%, 22%, 23%, 24%, 25%, 26%, 27%, 28%, 29% or 30% by weight of the composition. In some embodiments, the methylnaltrexone and lauryl sulfate ion pair may be present in an amount of at most about 10%, 11%, 12%, 13%, 14%, 15%, 16%, 17%, 18%, 19%, 20%, 21%, 22%, 23%, 24%, 25%, 26%, 27%, 28%, 29% or 30% by weight of the composition. In some embodiments, the methylnaltrexone and lauryl sulfate ion pair may be present in an amount of about 10%, 11%, 12%, 13%, 14%, 15%, 16%, 17%, 18%, 19%, 20%, 21%, 22%, 23%, 24%, 25%, 26%, 27%, 28%, 29% or 30% by weight of the composition.

In certain embodiments, where the pharmaceutical composition includes an ion pair of methylnaltrexone and docusate, the ion pair is present at about 1% to about 50%, about 10% to about 50%, about 15% to about 45%, about 20% to about 40%, about 15% to about 30%, or about 30% to about 45% by weight of the composition. For example, the methylnaltrexone and docusate ion pair may be present in an amount of at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45% by weight of the composition. In some embodiments, the methylnaltrexone and docusate ion pair may be present in an amount of at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45% by weight of the composition. In some embodiments, the methylnaltrexone and docusate ion pair may be present in an amount of about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45% by weight of the composition.

In a further aspect, a pharmaceutical composition in a liquid oral dosage form includes (i) methylnaltrexone, (ii) lauryl sulfate or docusate and (iii) one or more of an oil, surfactant, and a cosolvent, wherein the methylnaltrexone and lauryl sulfate or docusate are present in substantially equal molar amounts. As used herein, the term “substantially equal molar” means the moles of lauryl sulfate or docusate are within 5%, 4%, 3%, 2%, 1%, 0.1%, or 0.01% of the moles of methylnaltrexone. In one embodiment, the pharmaceutical composition includes lauryl sulfate. In another embodiment, the pharmaceutical composition includes docusate.

In one embodiment, the pharmaceutical composition includes methylnaltrexone and lauryl sulfate in an amount that is about 1% to about 75%, about 10% to about 60%, about 15% to about 50%, or about 20% to about 40% of the pharmaceutical composition by weight. In another embodiment, the pharmaceutical composition includes methylnaltrexone and docusate in an amount that is about 1% to about 75%, about 10% to about 60%, about 15% to about 50%, or about 20% to about 40% of the pharmaceutical composition by weight.

In some embodiments, the oil includes at least one of glyceryl monooleate, glyceryl monolinoleate, propylene glycol dicaprolate/dicaprate, soybean oil, polyglyceryl-3 dioleate, oleic acid, glyceryl caprylate, medium chain triglycerides, and a combination thereof. For example, in one embodiment, the oil includes glyceryl monolinoleate. In another exemplary embodiment, the oil includes oleic acid. In a further embodiment, the oil includes glyceryl caprylate. In a particular embodiment, the oil includes medium chain triglycerides. In a certain embodiment, the oil includes at least two oils, e.g., glyceryl caprylate and medium chain triglycerides. In some embodiments, the total oil content of the pharmaceutical composition is about 10% to about 80%, about 10% to about 20%, about 20% to about 50%, or about 50% to about 70% by weight of the composition.

In further embodiments, the pharmaceutical composition includes a surfactant. Suitable surfactants for use in the pharmaceutical compositions of the disclosure include oleoyl polyoxyl-6 glycerides, linoleoyl polyoxyl-6 glycerides, caprylocaproyl polyoxyl-8 glycerides, polysorbate 80, polyoxyl 40 hydrogenated castor oil, polyoxyl 15 hydroxystearate, lauroyl polyoxyl-32 glycerides, or a combination thereof. In an exemplary embodiment, the surfactant includes caprylocaproyl polyoxyl-8 glycerides. In another exemplary embodiment, the surfactant is polysorbate 80. In yet another exemplary embodiment, the surfactant is linoleoyl polyoxyl-6 glycerides. In some embodiments, the surfactant includes polyoxyl 40 hydrogenated castor oil. In some embodiments, the surfactant includes polyoxyl 15 hydroxystearate. In some embodiments, the surfactant includes lauroyl polyoxyl-32 glycerides. In some embodiments, the pharmaceutical composition includes about 10% to about 70%, about 15% to about 40%, or about 20% to about 35% of the surfactant by weight of the composition.

Suitable oils for use in the pharmaceutical compositions of the invention described herein include lipids and fatty acids that are derived from vegetable sources via esterification of fatty acids with alcohols, e.g., glycerol, polyglycerol, propylene glycol, and polyethylene glycol, and by the alcoholysis of vegetable oils and fats with glycerol, polyethylene glycol, and propylene glycol.

In some embodiments, oils suitable for inclusion in pharmaceutical compositions of the invention include, but are not limited to, glyceryl monooleate, glyceryl monolinoleate, propylene glycol dicaprolate/dicaprate, soybean oil, polyglyceryl-3 dioleate, oleic acid, glyceryl caprylate, medium chain triglycerides, and combinations thereof.

In a particular embodiment, the pharmaceutical composition includes glyceryl monooleate, e.g., Peceol™ available from Gattefossé, which includes mono-, di-, and triglycerides of oleic (C_{18:1}) acid, the monoester fraction being predominant. Glyceryl monooleate is used as a solubilizer for lipophilic active pharmaceutical ingredients (APIs). Glyceryl monooleate is also used in SEDDS and SMEDDS, as described herein.

Alternatively or in combination, the pharmaceutical compositions of the invention can include glyceryl monolinoleate. Glyceryl monolinoleate, e.g., Maisine® CC available from Gattefossé, is a winterized oil composed of long-chain mono, di- and triglycerides, primarily linoleic (C_{18:2}) and oleic acid (C_{18:1}). Glyceryl monolinoleate is used in lipid-based formulations to solubilize poorly water-soluble lipophilic APIs and is also used in self-emulsifying lipid formulations (SEDDS and SMEDDS). In some embodiments, the pharmaceutical compositions include glyceryl monolinoleate in an amount from about 3% to about 30%, from about 5% to about 25%, or from about 10% to about 20% by weight of the composition. In some embodiments, the pharmaceutical compositions include glyceryl monolinoleate in an amount of at least about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% by weight of the composition. In some embodiments, the pharmaceutical compositions include glyceryl monolinoleate in an amount of at most about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% by weight of the composition. In some embodiments, the pharmaceutical compositions include glyceryl monolinoleate in an amount of about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% by weight of the composition.

Alternatively, or in combination, the pharmaceutical compositions of the invention include propylene glycol. Propylene glycol dicaprolate/dicaprate, e.g., Labrafac™ PG available from Gattefossé, includes propylene glycol esters of caprylic (C₈) and capric (C₁₀) acids. Propylene glycol dicaprolate/dicaprate is also used in lipid-based formulations, SEDDS, and SMEDDS.

The pharmaceutical compositions of the invention described herein may further include medium chain triglycerides. Medium chain triglycerides, e.g., MIGLYOL® 812 available from IOI Oleo GmbH and Labrafac™ Lipophile WL 1349 available from Gattefossé, consists of medium-chain triglycerides of caprylic (C₈) and capric (C₁₀) acids. Medium chain triglycerides are also used in lipid-based formulations, SEDDS and SMEDDS. In some embodiments, the pharmaceutical compositions include medium chain triglycerides in an amount from about 3% to about 30%, from about 5% to about 20%, or from about 10% to about 15% by weight of the composition. In some embodiments, the pharmaceutical compositions include medium chain triglycerides in an amount of at least about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% by weight of the composition. In some embodiments, the pharmaceutical compositions include medium chain triglycerides in an amount of at most about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% by weight of the composition. In some embodiments, the pharmaceutical compositions include medium chain triglycerides in an amount of about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% by weight of the composition.

In further embodiments, the oil includes caprylic/capric triglyceride. Caprylic/capric triglyceride, e.g., MIGLYOL® 810 and MIGLYOL® 812 available from CREMER OLEO GmbH & Co. KG, are esters of caprylic and capric fatty acids and glycerin derived from saturated coconut and palm kernel oil.

In some embodiments, the oil includes a soybean oil-based excipient. Soybean oil-based excipients, e.g., Geloil™ SC available from Gattefossé, include a mixture of soybean oil, glyceryl distearate (C₁₈) and polyglyceryl-3 dioleate (C_{18:1}). Geloil™ SC serves as a vehicle to suspend pharmaceutical ingredients in soft gelatin capsule and has good dispersibility in aqueous fluid.

In some embodiments the oil includes polyglyceryl-3 dioleate. Polyglyceryl-3 dioleate, e.g., Plurol® Oleique CC 497, includes polyglyceryl-3-esters of oleic acid (C_{18:1}), the diester fraction being predominant. Polyglyceryl-3 dioleate also serves as a co-surfactant in SEDDS and SMEDDS formulations.

In some embodiments, the oil includes oleic acid. Oleic acid is a monounsaturated omega-9 fatty acid (C_{18:1}). In some embodiments, the pharmaceutical compositions include oleic acid in an amount from about 10% to about 40%, from about 15% to about 35%, or from about 20% to about 30% by weight of the composition. In some embodiments, the pharmaceutical compositions include oleic acid in an amount of at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% by weight of the composition. In some embodiments, the pharmaceutical compositions include oleic acid in an amount of at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% by weight of the composition. In some embodiments, the pharmaceutical compositions include oleic acid in an amount of about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% by weight of the composition.

In some embodiments, the oil includes glyceryl caprylate mono- and di-glycerides. Glyceryl caprylate mono- and diglycerides, e.g., IMWITOR® 988 and/or IMWITOR® 742 available from CREMER, includes a blend of glycerol esters of caprylic (C₈H₁₆O₂) acid derived from vegetable sources. In some embodiments, the pharmaceutical compositions include glyceryl caprylate mono- and diglycerides from about 10% to about 40%, from about 20% to about 35%, or from about 20% to about 30%, or about 30% to about 35% by weight of the composition. In alternative embodiments, the pharmaceutical compositions include glyceryl caprylate mono- and diglycerides in an amount from about 15% to about 45%, from about 25% to about 40%, or from about 30% to about 35% by weight of the composition. In some embodiments, the pharmaceutical compositions include glyceryl caprylate mono- and diglycerides in an amount of at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, or 45% by weight of the composition. In some embodiments, the pharmaceutical compositions include glyceryl caprylate mono- and diglycerides in an amount of at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, or 45% by weight of the composition. In some embodiments, the pharmaceutical compositions include glyceryl caprylate mono- and diglycerides in an amount of about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, or 45% by weight of the composition.

In some embodiments, the total oil content of the pharmaceutical composition is about 10% to about 80%, about 15% to about 70%, about 20% to about 60%, or about 30% to about 50% by weight of the composition. In some embodiments, the total oil content of the pharmaceutical composition is at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition. In some embodiments, the total oil content of the pharmaceutical composition is at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition. In some embodiments, the total oil content of the pharmaceutical composition is about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition.

In some embodiments, pharmaceutical compositions include one oil. In some embodiments, pharmaceutical compositions include two, three, four, five, or more oils. In an exemplary embodiment, the pharmaceutical composition includes two oils, e.g., medium chain triglycerides and glyceryl caprylate mono- and diglycerides. In another exemplary embodiment, the pharmaceutical composition includes three oils, e.g., medium chain triglycerides, glyceryl caprylate mono- and diglycerides, and oleic acid. In some embodiments, the pharmaceutical compositions do not include an oil as described herein.

Surfactants can be added to the pharmaceutical compositions disclosed herein, for example, to prepare self-emulsifying, self-microemulsifying drug delivery systems, and self-nanoemulsifying drug delivery systems.

Suitable surfactants for use in the pharmaceutical compositions of the invention described herein include oleoyl polyoxyl-6 glycerides, linoleoyl polyoxyl-6 glycerides, caprylocaproyl polyoxyl-8 glycerides, polysorbate 80, polyoxyl 40 hydrogenated castor oil, polyoxyl 15 hydroxystearate, lauroyl polyoxyl-32 glycerides, and combinations thereof.

In certain embodiments, the pharmaceutical compositions of the invention include oleoyl polyoxyl-6 glycerides. Oleoyl polyoxyl-6 glycerides, e.g., Labrafil® M 1944 CS available from Gattefossé, comprise mono-, di-, and triglycerides and PEG-6 (MW 300) mono- and diesters of oleic (C_{18:1}) acid. Oleoyl polyoxyl-6 glycerides are used to solubilize poorly-soluble APIs. Oleoyl polyoxyl-6 glycerides are also used in single excipient formulation systems to prepare SEDDS and can form SMEDDS when combined with high HLB surfactants, e.g., Labrasol® ALF or Gelucire® 44/14.

In some embodiments, the surfactant includes linoleoyl polyoxyl-6 glycerides. Linoleoyl polyoxyl-6 glycerides, e.g., Labrafil® M 2125 CS available from Gattefossé, comprise mono-, di-, and triglycerides and PEG-6 (MW 300) mono- and diesters for linoleic (C_{18:2}) acid. Linoleoyl polyoxyl-6 glycerides solubilize poorly water-soluble APIs in lipid-based formulations. Linoleoyl polyoxyl-6 glycerides also self-emulsify in aqueous media forming a coarse dispersion, i.e., SEDDS, and forms SMEDDS in pharmaceutical compositions that also include surfactants such as Labrasol® ALF or Gelucire® 44/14.

In some embodiments, the surfactant includes caprylocaproyl polyoxyl-8 glycerides. Caprylocaproyl polyoxyl-8 glycerides, e.g., Labrasol® ALF available from Gattefossé, comprise a small fraction of mono-, di- and triglycerides and mainly PEG-8 (MW 400) mono- and diesters of caprylic (C₈) and capric (C₁₀) acids. Caprylocaproyl polyoxyl-8 glycerides are a solubilizer for poorly-soluble APIs. Caprylocaproyl polyoxyl-8 glycerides are also used in single excipient formulation systems that self-emulsify in aqueous fluid into microemulsions (SMEDDS). In some embodiments, the pharmaceutical composition includes caprylocaproyl polyoxyl-8 glycerides in an amount from about 50% to about 80%, from about 55% to about 70%, or from about 60% to about 65% by weight of the composition. In some embodiments, the pharmaceutical composition includes caprylocaproyl polyoxyl-8 glycerides in an amount of at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition. In some embodiments, the pharmaceutical composition includes caprylocaproyl polyoxyl-8 glycerides in an amount of at most about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition. In some embodiments, the pharmaceutical composition includes caprylocaproyl polyoxyl-8 glycerides in an amount of about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition.

In some embodiments, the surfactant includes propylene glycol monolaurate. Propylene glycol monolaurate, e.g., Lauroglycol™ 90, includes propylene mono- and diesters of auric (Cu) acid, mainly monoesters with a small fraction of diesters. Propylene glycol monolaurate is used as a cosurfactant in SEDDS and SMEDDS.

In some embodiments, the surfactant includes propylene glycol monocaprylate. Propylene glycol monocaprylate, e.g., Capryol™ 90 available from Gattefossé, includes propylene glycol esters of acrylic acid (CO, primarily monoesters and a small fraction of diesters. Propylene glycol monocaprylate is a nonionic water-insoluble surfactant that is used as a cosurfactant in SEDDS and SMEDDS.

It should be appreciated that some embodiments of the pharmaceutical composition include one or more cosurfactants. For example, some embodiments of the pharmaceutical composition include up to 20% (w/w) cosurfactant (e.g., Capryol™ (propylene glycol caprylate) and/or Lauroglycol™ (Propylene glycol monolaurate)).

Suitable surfactants also include polysorbate 80 (e.g., TWEEN® 80 from Croda International Plc), polyoxyethylene sorbitan trioleate (e.g., TWEEN® 85 from Croda International Plc), PEG-35 castor oil, polyoxyl 40 hydrogenated castor oil (e.g., KOLLIPHOR® RH 40), polyoxyl 15 hydroxystearate (e.g., KOLLIPHOR® HS 15), lauroyl polyoxyl-32 glycerides (e.g., GELUCIRE® 44/14), and/or Vitamin E TPGS.

In some embodiments, the surfactant includes TWEEN® 80 from about 15% to about 50%, from about 20% to about 40%, from about 30% to about 35% by weight of the composition. In some embodiments, the composition includes TWEEN® 80 in amount of at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% by weight of the composition. In some embodiments, the composition includes TWEEN® 80 in amount of at most about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% by weight of the composition. In some embodiments, the composition includes TWEEN® 80 in an amount of about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% by weight of the composition.

In some embodiments, the surfactant includes polyoxyl 40 hydrogenated castor oil, e.g., KOLLIPHOR® RH40. In some embodiments, the polyoxyl 40 hydrogenated castor oil is KOLLIPHOR® RH 40. KOLLIPHOR® RH 40 is a digestible surfactant. In some embodiments, the pharmaceutical compositions include polyoxyl 40 hydrogenated castor oil in an amount from about 10% to about 80%, from about 20% to about 70%, or from about 25% to about 65% by weight of the composition. In alternative embodiments, the pharmaceutical compositions include polyoxyl 40 hydrogenated castor oil in an amount from about 15% to about 45%, from about 25% to about 40%, or from about 30% to about 40% by weight of the composition. In some embodiments, the pharmaceutical compositions include polyoxyl 40 hydrogenated castor oil in an amount of at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition. In some embodiments, the pharmaceutical compositions include polyoxyl 40 hydrogenated castor oil in an amount of at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition. In some embodiments, the pharmaceutical compositions include polyoxyl 40 hydrogenated castor oil in an amount of about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition.

In some embodiments, the surfactant includes polyoxyl 15 hydroxystearate from about 15% to about 50%, from about 20% to about 40%, from about 30% to about 35% by weight of the composition. In some embodiments, polyoxyl 15 hydroxy stearate is KOLLIPHOR® HS 15. KOLLIPHOR® HS 15 is a non-digestible surfactant. In some embodiments, the composition includes polyoxyl 15 hydroxystearate (e.g., KOLLIPHOR® HS 15) in amount of at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% by weight of the composition. In some embodiments, the composition includes polyoxyl 15 hydroxystearate (e.g., KOLLIPHOR® HS 15) in amount of at most about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% by weight of the composition. In some embodiments, the composition includes polyoxyl 15 hydroxystearate (e.g., KOLLIPHOR® HS 15) in amount of about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% by weight of the composition.

In some embodiments, the surfactant includes lauroyl polyoxyl-32 glycerides, e.g., GELUCIRE® 44/14. In some embodiments, the lauroyl polyoxyl-32 glycerides is GELUCIRE® 44/14. In some embodiments, the pharmaceutical compositions include lauroyl polyoxyl-32 glycerides in an amount from about 10% to about 80%, from about 20% to about 70%, or from about 25% to about 65% by weight of the composition. In alternative embodiments, the pharmaceutical compositions include lauroyl polyoxyl-32 glycerides in an amount from about 15% to about 45%, from about 25% to about 40%, or from about 30% to about 40% by weight of the composition. In some embodiments, the pharmaceutical compositions include lauroyl polyoxyl-32 glycerides in an amount of at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition. In some embodiments, the pharmaceutical compositions include lauroyl polyoxyl-32 glycerides in an amount of at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition. In some embodiments, the pharmaceutical compositions include lauroyl polyoxyl-32 glycerides in an amount of about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition.

In some embodiments, the pharmaceutical composition includes Vitamin E TPGS, which may behave as both a surfactant and a stabilizer. When used as a surfactant, Vitamin E TPGS may be provided in amount from about 10% to about 80% or from about 20% to about 70% by weight of the composition. In some embodiments, the pharmaceutical compositions include Vitamin E TPGS in an amount of at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition. In some embodiments, the pharmaceutical compositions include Vitamin E TPGS in an amount of at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition. In some embodiments, the pharmaceutical compositions include Vitamin E TPGS in an amount of about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition.

In some embodiments, the compositions described herein may include an additive. In some embodiments, the additive may be a stabilizer such as butylated hydroxytoluene (BHT), butylated hydroxyanisole (BHA), propyl gallate, ascorbic acid-6-palmitate, alpha tocopherol, Vitamin E TPGS (when provided as a stabilizer rather than a surfactant), or a combination thereof. In some embodiments, the stabilizer (e.g., BHT) may be provided in an amount of about 0.01% to about 10% by weight of the composition. In some embodiments, the stabilizer (e.g., BHT) may be provided in an amount of at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10% by weight of the composition. In some embodiments, the stabilizer (e.g., BHT) may be provided in an amount of at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10% by weight of the composition. In some embodiments, the stabilizer (e.g., BHT) may be provided in an amount of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10% by weight of the composition.

For example, butylated hydroxyanisole (BHA) may be provided in an amount of up to about 0.05% by weight of the composition. Propyl gallate may be provided in an amount of up to about 0.1% by weight of the composition. Ascorbic acid-6-palmitate may be provided in an amount of up to about 3% by weight of the composition. Alpha tocopherol may be provided in an amount of up to about 4% by weight of the composition.

As described herein, in some embodiments, the pharmaceutical compositions may include a cosolvent. In some embodiments, the cosolvent may be triacetin, ethanol, glycerol, propylene glycol, polyethylene glycol (e.g., PEG-400), or a combination thereof. In some embodiments, the cosolvent includes ethanol. In some embodiments, the cosolvent (e.g., ethanol) may be provided in an amount of about 1% to about 20%, or about 1% to about 10%, or about 5% to about 15% by weight of the composition. In some embodiments, the cosolvent (e.g., ethanol) may be provided in an amount of at least about 1, 2, 3, 4, 5, 6, 7, 8, 9, 10, 11, 12, 13, 14, 15, 16, 17, 18, 19, or 20% by weight of the composition. In some embodiments, the cosolvent (e.g., ethanol) may be provided in an amount of at most about 1, 2, 3, 4, 5, 6, 7, 8, 9, 10, 11, 12, 13, 14, 15, 16, 17, 18, 19, or 20% by weight of the composition. In some embodiments, cosolvent (e.g., ethanol) may be provided in an amount of about 1, 2, 3, 4, 5, 6, 7, 8, 9, 10, 11, 12, 13, 14, 15, 16, 17, 18, 19, or 20% by weight of the composition.

In some embodiments, the pharmaceutical compositions disclosed herein are formulated for enteric delivery. Enteric drug delivery vehicles, e.g., coatings, capsules, and other encapsulation technologies, are used to protect acid sensitive APIs from the stomach's low pH environment, to protect the stomach from irritating APIs, and to facilitate colonic drug delivery.

The delayed release of drugs from enteric formulations arises from the insolubility of enteric polymers at low pH values. Enteric polymers dissolve at a pH values of about 5.0-5.5 and higher. Enteric formulations can also be affected by factors, such as the nature of the API (e.g., whether the API is ionic), the thickness of the coating/capsule shell, the presence of imperfections (e.g., cracks, holes, etc.), the properties of the polymer(s) used (e.g., dissolution rate at relevant pHs), and agitation rate.

Enteric polymers, for use in the invention described herein, include polyacids, such as cellulose acetate phthalate, cellulose acetate trimellitate, polyvinyl acetate phthalate, hydroxypropylmethylcellulose phthalate, hydroxypropylmethylcellulose acetate succinate, methacrylate-ethylacrylate copolymers, and methacrylate-methylmethacrylate copolymers. To target the colon, a combination of pH-triggered (e.g., at pH 6.8-7.2) and enzyme-triggered polymers can be used. Additionally, capsules-in-capsules and coated or uncoated capsules including liquid filled hard capsules can be used to target colonic delivery.

Suitable enteric capsules for use in the pharmaceutical compositions of the disclosure include gelatin and EUDRAGIT® L 100-based capsules as described in U.S. Pat. No. 8,685,445 and hydroxypropyl methylcellulose acetate succinate-based capsules as described in US 20130295188A1. Enteric coated capsules are also contemplated. See, e.g., U.S. Pat. Nos. 4,518,433, 4,816,259, and 5,330,759. In some embodiments, enteric capsules are Vcaps® Enteric Capsules from Capsugel.

Enteric coated methylnaltrexone formulations have yielded unpredictable results. For example, while an enteric coated methylnaltrexone formulation more effectively reduced oral-cecal delay caused by morphine than an uncoated formulation (laxation data was not reported) (see, e.g., U.S. Pat. No. 6,274,591), capsules containing enterically coated spheroids of a formulation of methylnaltrexone surprisingly did not induce laxation in patients suffering from opioid-induced constipation (see, e.g., U.S. Pat. No. 8,524,276).

In some embodiments, the pharmaceutical composition described herein may include one or more of the liquid oral dosage forms described herein below.

In some embodiments, the pharmaceutical composition may be described as a methylnaltrexone lauryl sulfate ion pair formulation according to the following formula:

| | % (w/w) |
|---|---|
| Methylnaltrexone - Lauryl Sulfate | 20 |
| Labrasol ® | 64 |
| Maisine ® CC | 16 |
| Total | 100 |

Labrasol® and Maisine® CC were mixed according to the proportions listed above. Methylnaltrexone-Lauryl Sulfate was added and the mixture was incubated at about 60° C. in a water bath with mixing for 6 hours until the pharmaceutical composition was obtained as a single phase. Emulsions were prepared by emulsifying 0.5 ml of the pharmaceutical composition in 25 ml of 100 mM phosphate buffer pH 6.8. Mean droplet size and polydispersity index (PDI) were measured via dynamic light scattering. The mean droplet size was about 158 nm, and the PDI was 0.16. The emulsion was also visually examined for any precipitation or phase separation after 12 hours and was found to be stable without any precipitation or phase separation. The pharmaceutical composition was filled in size 00 hard gelatin capsules. Dissolution rate was measured in pH 2 and pH 6.8 media using a USP dissolution apparatus 2 by visual observation of shell dissolution. Capsule shells completely dissolved and released the pharmaceutical composition within 10 mins in both media.

In some embodiments, the pharmaceutical composition may be described as a methylnaltrexone docusate ion pair formulation according to the following formula:

| | % (w/w) |
|---|---|
| Methylnaltrexone - Docusate | 37 |
| Medium chain triglycerides | 10.71 |
| (MIGLYOL ® 812) | |
| IMWITOR ® 988 | 26.46 |
| Oleic acid | 25.83 |
| Total | 100 |

Medium chain triglycerides, IMWITOR® 988, and oleic acid were mixed according to the proportions listed above. Methylnaltrexone-Docusate was added and incubated at about 60° C. in a water bath with mixing for 6 hours until the pharmaceutical composition was obtained as a single phase. Emulsions were prepared by emulsifying 0.5 ml of the pharmaceutical composition in 25 ml of 100 mM phosphate buffer pH 6.8. Mean droplet size and PDI were measured via dynamic light scattering. The mean droplet size was about 300 nm, and the PDI was 0.45. The emulsion was visually examined for any precipitation or phase separation. The emulsion was stable up to 2 hours, then precipitation was observed.

In some embodiments, the pharmaceutical composition may be described as a methylnaltrexone docusate ion pair formulation according to the following formula:

| | % (w/w) |
|---|---|
| Methylnaltrexone - Docusate | 23 |
| Medium chain triglycerides | 13.09 |
| (MIGLYOL ® 812) | |
| IMWITOR ® 988 | 32.34 |
| TWEEN ® 80 | 31.57 |
| Total | 100 |

Medium chain triglycerides, IMWITOR® 988, and TWEEN®80 were mixed according to the proportions listed above. Methylnaltrexone-Docusate was added and incubated at about 60° C. in a water bath with mixing for 12 hours until the pharmaceutical composition was obtained as a single phase. Emulsions were prepared by emulsifying 0.5 ml of the pharmaceutical composition in 25 ml of 100 mM phosphate buffer pH 6.8. Mean droplet size and PDI were measured via dynamic light scattering. The mean droplet size was about 135 nm, and the PDI was 0.27. The emulsion was visually examined for any precipitation or phase separation and was found to be stable without any precipitation or phase separation for up to 12 hours. The pharmaceutical composition was filled in size 0 hard gelatin capsules, which were each then enclosed in size 00 Vcaps® Enteric Capsules. The capsule-in-capsule delivery vehicle was employed, because enteric capsules are not intended for liquid fill and were found to be incompatible with IMWITOR®988. Dissolution rate was measured in pH 2 and pH 6.8 media using a USP dissolution apparatus 2 by visual observation of shell dissolution. Capsule shells did not disintegrate after 2 hours in pH 2 media and completely disintegrated and released the pharmaceutical composition within 7 mins in pH 6.8 media.

In some embodiments, the pharmaceutical composition may be described as a methylnaltrexone docusate ion pair formulation according to the following formula:

| | % (w/w) |
|---|---|
| Methylnaltrexone - Docusate | 23 |
| Medium chain triglycerides | 13.09 |
| (MIGLYOL ® 812) | |
| IMWITOR ® 988 | 32.34 |
| TWEEN ® 80 | 31.57 |
| Total | 100 |

Medium chain triglycerides, IMWITOR® 988, and TWEEN® 80 were mixed according to the proportions listed above. Methylnaltrexone-Docusate was added and incubated at around 60° C. in a water bath under continuous mixing for 12 hours until the pharmaceutical composition was obtained as a single phase. Emulsions were prepared by emulsifying 0.5 ml of the pharmaceutical composition in 25 ml of 100 mM phosphate buffer pH 6.8. Mean droplet size and PDI were measured via dynamic light scattering. The mean droplet size was about 131 nm, and PDI was 0.16. The emulsion was visually examined for any precipitation or phase separation for 12 hours and was found to be stable without any precipitation or phase separation. The pharmaceutical composition was filled in size 00 hard gelatin capsules. Dissolution rate was measured in pH 2 and pH 6.8 media in USP dissolution apparatus 2 by visual observation of shell dissolution. Capsule shells completely disintegrated and released the pharmaceutical composition within 10 mins in both media.

In some embodiments, the pharmaceutical composition may be described as a formulation including methylnaltrexone-docusate, IMWITOR® 988, medium chain glyceride (MCT), TWEEN® 80, and a stabilizer (e.g., butylated hydroxytoluene), as shown below:

| | % (w/w) |
|---|---|
| Methylnaltrexone - Docusate | 10.00 |
| IMWITOR ® 988 | 37.80 |
| Medium Chain Glyceride (MCT) | 13.45 |
| TWEEN ® 80 | 38.70 |
| Butylated Hydroxytoluene (BHT) | 0.05 |
| Total | 100 |

In some embodiments, the pharmaceutical composition may be described as a formulation including methylnaltrexone-docusate, IMWITOR® 988, medium chain glyceride (MCT), TWEEN® 80, and a stabilizer (e.g., butylated hydroxytoluene), as shown below:

| | % (w/w) |
|---|---|
| Methylnaltrexone - Docusate | 25.00 |
| IMWITOR ® 988 | 31.50 |
| Medium Chain Glyceride (MCT) | 11.20 |
| TWEEN ® 80 | 32.25 |
| Butylated Hydroxytoluene (BHT) | 0.05 |
| Total | 100 |

In some embodiments, the pharmaceutical composition may be described as a formulation including methylnaltrexone-docusate, ethanol, KOLLIPHOR® RH 40, and a stabilizer (e.g., butylated hydroxytoluene), as shown below:

| | % (w/w) |
|---|---|
| Methylnaltrexone - Docusate | 25.00 |
| Ethanol | 8.33 |
| Vitamin E TPGS | 5.00 |
| KOLLIPHOR ® RH 40 | 61.62 |
| Butylated Hydroxytoluene (BHT) | 0.05 |
| Total | 100 |

In some embodiments, the pharmaceutical composition may be described as a formulation including methylnaltrexone-docusate, ethanol, KOLLIPHOR® RH 40, KOLLIPHOR® HS 15, and a stabilizer (e.g., butylated hydroxytoluene), as shown below:

| | % (w/w) |
|---|---|
| Methylnaltrexone - Docusate | 25.00 |
| Ethanol | 8.33 |
| Vitamin E TPGS | 5.00 |
| KOLLIPHOR ® RH 40 | 30.81 |
| KOLLIPHOR ® HS 15 | 30.81 |
| Butylated Hydroxytoluene (BHT) | 0.05 |
| Total | 100 |

In some embodiments, the pharmaceutical composition may be described as a formulation including methylnaltrexone-docusate, ethanol, Vitamin E TPGS, and a stabilizer (e.g., butylated hydroxytoluene), as described below:

| | % (w/w) |
|---|---|
| Methylnaltrexone - Docusate | 25.00 |
| Ethanol | 8.33 |
| Vitamin E TPGS | 66.62 |
| (as a non-ionic surfactant) | |
| Butylated Hydroxytoluene (BHT) | 0.05 |
| Total | 100 |

In some embodiments, the pharmaceutical composition may be described as a formulation including methylnaltrexone-docusate, ethanol, GELUCIRE® 44/14, and a stabilizer (e.g., butylated hydroxytoluene), as shown below:

| | % (w/w) |
|---|---|
| Methylnaltrexone - Docusate | 25.00 |
| Ethanol | 8.33 |
| GELUCIRE ® 44/14 (lauroyl | 66.62 |
| polyoxyl-32 glycerides NF) | |
| Butylated Hydroxytoluene (BHT) | 0.05 |
| Total | 100 |

### Methylnaltrexone Pharmaceutical Compositions for Parenteral and Subcutaneous Administration

In some embodiments, the methods described herein include the parenteral administration of methylnaltrexone, for example, methylnaltrexone bromide, as, for example, a subcutaneous methylnaltrexone formulation. In some embodiments, a subcutaneous methylnaltrexone formulation may include methylnaltrexone, for example, methylnaltrexone bromide, a chelating agent such as a calcium salt chelating agent, a stabilizing agent, an isotonic agent, and a carrier. In some embodiments, the pH of the formulation is between about a pH of 2 to about a pH of 6.

In some embodiments, subcutaneous methylnaltrexone formulations may include methylnaltrexone, for example, methylnaltrexone bromide, together with one or more excipients, such as, for example, one or more chelating agents, a calcium ion, isotonic agents, carriers, buffers, co-solvents, diluents, preservatives, and/or surfactants, or combinations thereof. One skilled in the art will readily appreciate that the same ingredient can sometimes perform more than one function, or can perform different functions in the context of different formulations, and/or portions of a formulation, depending upon the amount of the ingredient and/or the presence of other ingredients and/or active compound(s).

In some embodiments, methylnaltrexone is provided in the methods described herein as subcutaneous RELISTOR® injectable (prefilled syringe or vial). In some embodiments, methylnaltrexone is provided in the methods described herein as one or more doses of subcutaneous RELISTOR® (8 mg/0.4 mL) injectable prefilled syringe or one or more doses of subcutaneous RELISTOR® (12 mg/0.6 mL) injectable prefilled syringe or vial.

In some embodiments, the subcutaneous methylnaltrexone, for example, methylnaltrexone bromide, formulation comprises a chelating agent. In some embodiments, a chelating agent may be present in an amount from about 0.01 mg/mL to about 2 mg/mL or about 0.1 mg/mL to about 1 mg/mL in the formulation, or about 0.2 mg/mL to about 0.8 mg/mL of the formulation. In some embodiments, a chelating agent may be present in an amount from about 0.2 mg/mL, about 0.3 mg/mL, about 0.4 mg/mL, about 0.5 mg/mL, or about 0.6 mg/mL, in the formulation.

Examples of chelating agents include, but are not limited to, ethylenediaminetetraacetic acid (also synonymous with EDTA, edetic acid, versene acid, and sequestrene), and EDTA derivatives, such as sodium EDTA, and potassium EDTA, diammonium EDTA, dipotassium EDTA, disodium EDTA, TEA-EDTA, tetrasodium EDTA, tripotassium EDTA, trisodium EDTA, HEDTA, and trisodium HEDTA, and related salts thereof. Other chelating agents include niacinamide and derivatives thereof and sodium desoxycholate and derivatives thereof, ethylene glycol-bis-(2-aminoethyl)-N,N,N′,N′-tetraacetic acid (EGTA) and derivatives thereof, diethylenetriaminepentaacetic acid (DTPA) and derivatives thereof, N,N-bis(carboxymethyl)glycine (NTA) and derivatives thereof, nitrilotriacetic acid and derivatives thereof. Still other chelating agents include citric acid and derivatives thereof. Citric acid also is known as citric acid monohydrate. Derivatives of citric acid include anhydrous citric acid and trisodiumcitrate-dihydrate. In some embodiments, chelating agent is selected from EDTA or an EDTA derivative or EGTA or an EGTA derivative. In some embodiments chelating agent is EDTA disodium such as, for example, EDTA disodium hydrate.

In some embodiments, a subcutaneous methylnaltrexone, for example, methylnaltrexone bromide, formulation comprises a calcium ion and a chelating agent included as a single component of the formulation. Thus, in some embodiments a calcium salt chelating agent may be present in an amount from about 0.01 mg/mL to about 2 mg/mL or about 0.1 mg/mL to about 1 mg/mL in the formulation, or about 0.2 mg/mL to about 0.8 mg/mL of the formulation. In some embodiments, calcium salt chelating agent may be present in an amount from about 0.2 mg/mL, about 0.3 mg/mL, about 0.4 mg/mL, about 0.5 mg/mL, or about 0.6 mg/mL, in the formulation.

Appropriate calcium salt chelating agents include any pharmaceutically acceptable chelating agents and calcium salts thereof. Common calcium salt chelating agents include, but are not limited to calcium ethylenediaminetetraacetic acid (EDTA) and calcium salt EDTA derivatives, calcium ethylene glycol-bis-(2-aminoethyl)-N,N,N′,N′-tetraacetic acid (EGTA) and calcium salt EGTA derivatives, calcium diethylenetriaminepentaacetic acid (DTPA) and calcium salt DTPA derivatives, calcium N,N-bis(carboxymethyl)glycine (NTA) and calcium salt NTA derivatives, and calcium citrate and derivatives thereof. In some embodiments, chelating agent is selected from calcium EDTA or a calcium salt EDTA derivative or calcium EGTA or a calcium salt EGTA derivative. In some embodiments, the chelating agent may be calcium EDTA disodium such as, for example, calcium EDTA disodium hydrate.

In some embodiments, a subcutaneous methylnaltrexone formulation comprises at least methylnaltrexone, for example, methylnaltrexone bromide, a calcium salt chelating agent and an isotonic agent. An isotonic agent useful in such formulations can be any pharmaceutically acceptable isotonic agent. Common isotonic agents include agents selected from the group consisting of sodium chloride, mannitol, lactose, dextrose (hydrous or anhydrous), sucrose, glycerol, and sorbitol, and solutions of the foregoing. In certain embodiments, the formulation comprises methylnaltrexone bromide, an isotonic agent which is sodium chloride, and a calcium salt chelating agent which is calcium EDTA or a calcium salt EDTA derivative. In some embodiments, the EDTA is calcium EDTA disodium.

Still additional components such as stabilizing agents, buffers, co-solvents, diluents, preservatives, and/or surfactants, and the like, may be included in subcutaneous methylnaltrexone formulations. In some embodiments, such formulations may contain additional agents which comprise from about 1% to about 30% or about 1% to about 12% of the formulation or about 1% to about 10%, based upon total weight of the formulation. In some embodiments, additional agents may comprise from about 1%, about 2%, about 5%, about 8% or about 10% of the formulation, based upon total weight of the formulation. Optionally included additional ingredients are described below.

In some embodiments, subcutaneous methylnaltrexone, for example, methylnaltrexone bromide, formulations comprise a stabilizing agent. In some embodiments, stabilizing agent may be present in an amount from about 0.01 mg/mL to about 2 mg/mL or about 0.05 mg/mL to about 1 mg/mL in the formulation, or about 0.1 mg/mL to about 0.8 mg/mL in the formulation. In some embodiments, stabilizing agent may be present in an amount from about 0.15 mg/mL, about 0.2 mg/mL, about 0.25 mg/mL, about 0.3 mg/mL, about 0.35 mg/mL, or about 0.4 mg/mL.

Suitable stabilizing agents for use in the subcutaneous methylnaltrexone formulations described herein include, but are not limited to glycine, benzoic acid, citric, glycolic, lactic, malic, and maleic acid. In some embodiments, such formulations comprise glycine. In some embodiments, glycine comprises glycine-HCl.

In certain embodiments, a stabilizing agent is added to the subcutaneous methylnaltrexone, for example, methylnaltrexone bromide, formulation in an amount sufficient to adjust and maintain the pH of the formulation. Thus, in some embodiments, a stabilizing agent acts as a buffer function in addition to its role as a stabilizer. In some embodiments, a stabilizing agent may act as a buffer agent, so as to maintain the pH of the formulation. In certain embodiments, the pH is between about pH 2.0 and about pH 6.0. In some embodiments, the pH of the formulation is between about pH 2.6 and about pH 5.0. In some embodiments, the pH of the formulation is between about pH 3.0 and about pH 4.0. In some embodiments, the pH of the formulation is between about pH 3.4 and about pH 3.6. In some embodiments, the pH of the formulation is about pH 3.5.

In some embodiments, subcutaneous methylnaltrexone formulations that may be used in the methods described herein may comprise methylnaltrexone bromide, calcium EDTA or a calcium salt EDTA derivative, water for injection, sodium chloride, glycine HCl, and the pH of the formulation is between about pH 3.0 and about pH 4.0. In some embodiments, such formulations comprise methylnaltrexone, for example, methylnaltrexone bromide, calcium EDTA or a calcium salt EDTA derivative, water for injection, sodium chloride, glycine HCl, and the pH of the formulation is between about pH 3.4 and about pH 3.6. In some embodiments, such formulations comprise methylnaltrexone bromide, calcium EDTA disodium, water for injection, sodium chloride, and glycine HCl, and the formulation has a pH of about 3.5. In certain embodiments, the pH is adjusted with glycine. In some embodiments, glycine is glycine HCl.

### Combination Products and Combined Administration

It will also be appreciated that the pharmaceutical compositions provided herein can be employed in combination therapies, that is, provided pharmaceutical compositions can be administered concurrently with, prior to, or subsequent to, one or more other desired therapeutics or medical procedures. Particular combination therapies (therapeutics or procedures) to employ in a combination regimen will take into account compatibility of the desired therapeutics and/or procedures and the desired therapeutic effect to be achieved. It will also be appreciated that therapies employed may achieve a desired effect for the same disorder (for example, a formulation may be administered concurrently with another compound used to treat the same disorder), or they may achieve different effects (e.g., control of any adverse effects). As used herein, additional therapeutic compounds which are normally administered to treat or prevent a particular disease, or condition, are known as “appropriate for the disease, or condition, being treated.”

In certain embodiments, pharmaceutical compositions of the disclosure and one or more other active agents may be administered together in a single formulation (e.g., unit dosage form); in other embodiments, pharmaceutical compositions and one or more other active agents may be administered as separate pharmaceutical compositions. In certain embodiments, methylnaltrexone, or a salt thereof, or an ion pair thereof, and/or one or more other active agents may be administered in multiple doses.

In other embodiments, the other active agent administered in combination with methylnaltrexone or formulation of the invention is an anticancer agent, optionally, wherein the anticancer agent is selected from the group consisting of a chemotherapeutic agent, an immunotherapy agent, a hormone therapy, a targeted drug therapy, a stem cell therapy, a radiotherapy, a cyroablation therapy, and an anti-angiogenic agent. In certain embodiments, methylnaltrexone or formulation of the invention is administered in combination with surgery. In certain embodiments, methylnaltrexone or formulation of the invention is administered in combination with a bone marrow transplant.

In other embodiments, the other active agent administered in combination with methylnaltrexone or formulation of the invention is an opioid. Combination therapy of methylnaltrexone and an opioid can allow simultaneous relief of pain and minimization of opioid-associated side effects (e.g., gastrointestinal effects, such as delayed gastric emptying, and altered GI tract motility). Accordingly, in certain embodiments, the invention described herein provides a unit dosage form comprising a combination of methylnaltrexone, or a salt thereof, or an ion pair thereof, with an opioid together. In some embodiments, a combination of methylnaltrexone, or a salt thereof, with an opioid together is provided in a liquid oral dosage form (e.g., a capsule) suitable for oral administration.

Therapeutic opioids, including those used for analgesia, are known in the art. For example, opioid compounds include, but are not limited to, alfentanil, anileridine, asimadoline, bremazocine, burprenorphine, butorphanol, codeine, dezocine, diacetylmorphine (heroin), dihydrocodeine, diphenoxylate, ethylmorphine, fedotozine, fentanyl, funaltrexamine, hydrocodone, hydromorphone, levallorphan, levomethadyl acetate, levorphanol, loperamide, meperidine (pethidine), methadone, morphine, morphine-6-glucoronide, nalbuphine, nalorphine, nicomorphine, opium, oxycodone, oxymorphone, papaveretum, pentazocine, propiram, propoxyphene, remifentanyl, sufentanil, tilidine, trimebutine, and tramadol. In some embodiments the opioid is at least one opioid selected from alfentanil, buprenorphine, butorphanol, codeine, dezocine, dihydrocodeine, fentanyl, hydrocodone, hydromorphone, levorphanol, meperidine (pethidine), methadone, morphine, nalbuphine, nicomorphine, oxycodone, oxymorphone, papaveretum, pentazocine, propiram, propoxyphene, sufentanil and/or tramadol. In certain embodiments of the invention described herein, the opioid is selected from morphine, codeine, oxycodone, hydrocodone, dihydrocodeine, propoxyphene, fentanyl, tramadol, and mixtures thereof. In a particular embodiment, the opioid is loperamide. In other embodiments, the opioid is a mixed agonist such as butorphanol. In some embodiments, the subjects are administered more than one opioid, for example, morphine and heroin or methadone and heroin.

Typically, the amount of other active agent(s) administered in combination therapy may be no more than the amount that would normally be administered in monotherapy with the relevant agent(s). In certain embodiments, the amount of other active agent administered in combination therapy may be less than that normally administered in monotherapy with the relevant agent(s). For example, in certain embodiments of the invention described herein, the amount of additional active agent can range from about 50% to about 100% of the amount normally present in a formulation comprising that compound as the only therapeutic agent.

In certain embodiments, pharmaceutical compositions may also be used in conjunction with and/or in combination with conventional therapies for gastrointestinal dysfunction to aid in the amelioration of constipation and bowel dysfunction. For example, conventional therapies include, but may not be limited to functional stimulation of the intestinal tract, stool softening agents, laxatives (e.g., diphelymethane laxatives, cathartic laxatives, osmotic laxatives, saline laxatives), bulk forming agents and laxatives, lubricants, intravenous hydration, and nasogastric decompression.

### Uses and Kits of Pharmaceutical Compositions

The invention described herein provides pharmaceutically acceptable compositions comprising methylnaltrexone or a salt thereof or an ion pair thereof for reducing mortality in a subject who may be suffering from an underlying disease or condition. The delivery of such pharmaceutical compositions can be in any context, and by any route, in which such delivery is desirable. In certain embodiments, provided pharmaceutical compositions are useful for the delivery of methylnaltrexone or a salt thereof or an ion pair thereof in antagonizing undesirable side effects of opioid therapy (e.g., increased risk of mortality) in subjects and, particularly, in subjects suffering from an underlying disease or condition. Furthermore, pharmaceutical compositions may be used to treat subjects who are treated with opioid therapies, such as opioid analgesic therapy, and who may have an increased mortality risk. In certain embodiments of the invention described herein, the methods are for use in human subjects.

Without being limited to any one theory of the invention, it has been surprisingly discovered that MNTX therapy leads to a reduction in all-cause mortality risk in subjects receiving opioid therapy. Indeed, compared to placebo treated subjects, it has been found that MNTX therapy resulted in a reduction of all-cause mortality risk by about 60%. However, this reduction in all-cause mortality risk with MNTX therapy appeared significant for certain subject populations as compared to others where it did not lead to a significant reduction in all-cause mortality risk. For example, it was surprisingly found that MNTX therapy, as described herein, led to significant decreases in all-cause mortality risk for those subjects that (a) had cancer (i.e., about 53% risk reduction); (b) were women (i.e., about 66% risk reduction); (c) were less than 60 years of age (i.e., about 79% reduction); or (d) had a chronic condition (i.e., about 68% risk reduction). Equipped with these surprising discoveries, the invention described herein demonstrates a treatment of certain subject populations having an increased risk of all-cause mortality due to opioid therapy whereby administration of MNTX to a subject of the subject population, by way of one or more pharmaceutical compositions described herein, results in a reduction of all-cause mortality risk.

Accordingly, in an embodiment, the invention includes a method for reducing mortality risk in a subject, wherein the subject may be receiving opioid therapy and/or suffering from an underlying disease or condition, comprising administering a pharmaceutical composition described herein to the subject. In some embodiments, the subject may be receiving opioid therapy. In some embodiments, the subject may be suffering from an underlying disease or condition. In some embodiments, the subject may be receiving opioid therapy and suffering from an underlying disease or condition.

In an embodiment, the invention includes a method for reducing mortality risk in a subject, wherein the subject is receiving opioid therapy and is not suffering from an underlying disease or condition, comprising administering a pharmaceutical composition described herein to the subject.

In an embodiment, the invention includes a method for reducing mortality risk in a subject, wherein the subject is receiving opioid therapy and is suffering from, and/or has previously suffered from, opioid-induced bowel dysfunction (OIBD) or opioid induced constipation (OIC), comprising administering a pharmaceutical composition described herein to the subject.

In an embodiment, the invention includes a method for reducing mortality risk in a subject, wherein the subject is receiving opioid therapy but is not suffering from, and/or has not previously suffered from, opioid-induced bowel dysfunction (OIBD) or opioid induced constipation (OIC), comprising administering a pharmaceutical composition described herein to the subject.

In an embodiment, the invention includes a method of ameliorating opioid analgesic related mortality risk in a subject in need thereof, comprising administering a pharmaceutical composition described herein to the subject, wherein the subject is a woman, is less than 60 years of age, has cancer, and/or has a chronic condition (i.e., an advanced illness, a cancer, and/or a chronic non-cancer pain). In some embodiments, the subject does not have, and/or has not previously suffered from, opioid-induced bowel dysfunction (OIBD) or opioid induced constipation (OIC).

In an embodiment, the invention includes a method of reducing mortality risk in a subject being treated with opioid therapy, the method comprising the steps of: (a) determining whether the subject has, and/or has previously had, an increased risk of mortality based on the presence of various risk factors, for example, as listed under Table 3 (e.g., a cardiovascular risk factor); (b) determining whether the subject has cancer, is a woman, is less than 60 years of age, and/or has a chronic condition; and (c) administering a pharmaceutical composition to the subject.

In an embodiment, the invention includes a method of reducing mortality risk in a subject being treated with opioid therapy (wherein the subject does not have and/or has not previously suffered from OIBD or OIC), the method comprising the steps of: (a) determining whether the subject has, and/or has previously had, an increased risk of mortality based on the presence of various risk factors, for example, as listed under Table 3 (e.g., a cardiovascular risk factor); (b) determining whether the subject has cancer, is a woman, is less than 60 years of age, and/or has a chronic condition; and (c) administering a pharmaceutical composition to the subject.

In an embodiment, the invention includes a method of reducing mortality risk in a subject being treated with opioid therapy and having at least one risk factor for increased mortality (see, e.g., the risk factors described in Table 3), the method comprising the steps of: (a) determining whether the subject has cancer, is a woman, is less than 60 years of age, and/or has a chronic condition; and (b) administering a pharmaceutical composition to the subject.

In an embodiment, the invention includes a method of reducing mortality risk in a subject being treated with opioid therapy, the method comprising the steps of: (a) determining whether the subject has, and/or has previously had, an increased risk of mortality based on the presence of various risk factors, for example, as listed under Table 3 (e.g., a cardiovascular risk factor); and (b) administering a pharmaceutical composition to the subject; wherein the subject has cancer, is a woman, is less than 60 years of age, and/or has a chronic condition.

In an embodiment, the invention includes a method of reducing mortality risk in a subject having an underlying disease or condition being treated with opioid therapy, the method comprising the steps of: (a) determining whether the subject has, and/or has previously had, an increased risk of mortality based on the presence of various risk factors, for example, as listed under Table 3 (e.g., a cardiovascular risk factor); (b) determining whether the subject has cancer, is a woman, is less than 60 years of age, and/or has a chronic condition; and (c) administering a pharmaceutical composition to the subject.

In an embodiment, the invention includes a method of reducing mortality risk in a subject having an underlying disease or condition being treated with opioid therapy (wherein the subject does not have and/or has not previously suffered from OIBD or OIC), the method comprising the steps of: (a) determining whether the subject has, and/or has previously had, an increased risk of mortality based on the presence of various risk factors, for example, as listed under Table 3 (e.g., a cardiovascular risk factor); (b) determining whether the subject has cancer, is a woman, is less than 60 years of age, and/or has a chronic condition; and (c) administering a pharmaceutical composition to the subject.

In an embodiment, the invention includes a method of reducing mortality risk in a subject having an underlying disease or condition being treated with opioid therapy and having at least one risk factor for increased mortality (see, e.g., the risk factors described in Table 3), the method comprising the steps of: (a) determining whether the subject has cancer, is a woman, is less than 60 years of age, and/or has a chronic condition; and (b) administering a pharmaceutical composition to the subject.

In an embodiment, the invention includes a method of reducing mortality risk in a subject having an underlying disease or condition being treated with opioid therapy, the method comprising the steps of: (a) determining whether the subject has, and/or has previously had, an increased risk of mortality based on the presence of various risk factors, for example, as listed under Table 3 (e.g., a cardiovascular risk factor); and (b) administering a pharmaceutical composition to the subject; wherein the subject has cancer, is a woman, is less than 60 years of age, and/or has a chronic condition.

In some embodiments of the foregoing methods, the underlying disease or condition may be an acute condition or a chronic condition. In some embodiments, the underlying disease or condition is an acute condition. In some embodiments, the underlying disease or condition is a chronic condition. In some embodiments, the acute condition is post-operative ileus. In some embodiments, the chronic condition is selected from the group consisting of an advanced illness, a cancer, a chronic non-cancer pain, and combinations thereof.

In some embodiments of the foregoing methods, the underlying disease or condition is selected from the group consisting of an advanced illness, cancer, a chronic non-cancer pain, postoperative ileus, recovery from orthopedic surgery, and combinations thereof.

In some embodiments of the foregoing methods, the cancer may include a carcinoma, a sarcoma, a melanoma, a leukemia, and/or a blastoma. In some embodiments, the cancer may be selected from the group consisting of a breast cancer, a liver cancer, a head and neck cancer, an esophageal cancer, a stomach cancer, a small intestine cancer, a colon cancer, a rectal cancer, an anal cancer, a skin cancer, a glandular cancer, a circulatory cancer, a prostate cancer, a pancreatic cancer, a hematopoietic cancer, a bone marrow cancer, a bone cancer, a cartilage cancer, a fat cancer, a brain cancer, a nerve cancer, a lung cancer, and a lymph cancer.

In another embodiment, the subject is not suffering from a cancer.

In some embodiments of the foregoing methods, the subject is a female subject.

In some embodiments of the foregoing methods, the subject is less than 60 years of age, or less than 50 years of age, or less than 40 years of age, or less than 30 years of age.

In some embodiments of the foregoing methods, the subject has a risk factor, such as a cardiovascular risk factor which may be one or more of hyperlipidemia/hypercholesterolemia, diabetes mellitus, stroke, myocardial infarction, hypertension, and angina.

In some embodiments of the foregoing methods, the subject is receiving (or has received) opioid therapy, which may include administration of at least one opioid to the subject. In some embodiments, the subject may have received or is receiving at least about 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, or 80000 mg of oral morphine equivalents (OME) per day. In some embodiments, the subject may have received or is receiving at most about 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, or 80000 mg of oral morphine equivalents (OME) per day. In some embodiments, the subject may have received or is receiving about 1 mg to about 1000 mg, or about 10 mg to about 500 mg, or about 20 mg to about 300 mg, or about 25 to about 200 mg of oral morphine equivalents (OME) per day.

In some embodiments of the foregoing methods, the at least one opioid may include one or more of alfentanil, anileridine, asimadoline, bremazocine, burprenorphine, butorphanol, codeine, dezocine, diacetylmorphine (heroin), dihydrocodeine, diphenoxylate, ethylmorphine, fedotozine, fentanyl, funaltrexamine, hydrocodone, hydromorphone, levallorphan, levomethadyl acetate, levorphanol, loperamide, meperidine (pethidine), methadone, morphine, morphine-6-glucoronide, nalbuphine, nalorphine, nicomorphine, opium, oxycodone, oxymorphone, papaveretum, pentazocine, propiram, propoxyphene, remifentanyl, sufentanil, tilidine, trimebutine, and tramadol. In some embodiments the opioid is at least one opioid selected from alfentanil, buprenorphine, butorphanol, codeine, dezocine, dihydrocodeine, fentanyl, hydrocodone, hydromorphone, levorphanol, meperidine (pethidine), methadone, morphine, nalbuphine, nicomorphine, oxycodone, oxymorphone, papaveretum, pentazocine, propiram, propoxyphene, sufentanil and/or tramadol.

In an embodiment, the invention includes a method for reducing mortality risk in a subject receiving opioid therapy (as described herein) comprising administering one or more of the pharmaceutical compositions described herein. In some embodiments, the subject is suffering from an underlying disease or condition. In some embodiments, the method for reducing mortality risk in a subject receiving opioid therapy comprises administering a therapeutically effective amount of one or more of the pharmaceutical compositions described herein. In some embodiments, the pharmaceutical composition may be one or more of a methylnaltrexone pharmaceutical composition for oral administration as described herein, a methylnaltrexone ion pair pharmaceutical composition as described herein, and a methylnaltrexone pharmaceutical composition for parenteral and subcutaneous administration as described herein. In some embodiments, the underlying disease or condition may be selected from the group consisting of an advanced illness, cancer, a chronic non-cancer pain, postoperative ileus, and recovery from orthopedic surgery, and combinations thereof.

In an embodiment, the invention includes a method for reducing mortality risk in a subject with a cancer and receiving opioid therapy (as described herein) comprising administering one or more of the pharmaceutical compositions described herein. In some embodiments, the method for reducing mortality risk in a subject receiving opioid therapy comprises administering a therapeutically effective amount of one or more of the pharmaceutical compositions described herein. In some embodiments, the pharmaceutical composition may be one or more of a methylnaltrexone pharmaceutical composition for oral administration as described herein, a methylnaltrexone ion pair pharmaceutical composition as described herein, and a methylnaltrexone pharmaceutical composition for parenteral and subcutaneous administration as described herein.

In an embodiment, the invention includes a method for reducing mortality risk in a subject less than 60 years of age receiving opioid therapy (as described herein) comprising administering one or more of the pharmaceutical compositions described herein. In some embodiments, the subject is suffering from an underlying disease or condition. In some embodiments, the method for reducing mortality risk in a subject receiving opioid therapy comprises administering a therapeutically effective amount of one or more of the pharmaceutical compositions described herein. In some embodiments, the pharmaceutical composition may be one or more of a methylnaltrexone pharmaceutical composition for oral administration as described herein, a methylnaltrexone ion pair pharmaceutical composition as described herein, and a methylnaltrexone pharmaceutical composition for parenteral and subcutaneous administration as described herein. In some embodiments, the underlying disease or condition may be selected from the group consisting of an advanced illness, cancer, a chronic non-cancer pain, postoperative ileus, and recovery from orthopedic surgery, and combinations thereof. In some embodiments, the subject is less than 50 years of age, or less than 40 years of age, or less than 30 years of age.

In an embodiment, the invention includes a method for reducing mortality risk in a subject with a chronic condition receiving opioid therapy (as described herein) comprising administering one or more of the pharmaceutical compositions described herein. In some embodiments, the method for reducing mortality risk in a subject receiving opioid therapy comprises administering a therapeutically effective amount of one or more of the pharmaceutical compositions described herein. In some embodiments, the pharmaceutical composition may be one or more of a methylnaltrexone pharmaceutical composition for oral administration as described herein, a methylnaltrexone ion pair pharmaceutical composition as described herein, and a methylnaltrexone pharmaceutical composition for parenteral and subcutaneous administration as described herein. In some embodiments, the chronic condition may be selected from the group consisting of an advanced illness, cancer, a chronic non-cancer pain, and combinations thereof.

In some embodiments of the foregoing methods, the pharmaceutical composition described herein may be administered intermittently, QD, BID, TID, or QID. In some embodiments, the pharmaceutical composition may be administered at least every 6 hours, at least every 12 hours, at least once a day (e.g., as a single dose), or at least once every other day, optionally, wherein the composition is administered at least once a day (QD), at least twice a day (BID), at least three times a day (TID), at least four times a day (QID), or continuously. In certain embodiments, the subject is administered the pharmaceutical composition as described herein at least once a day. In certain embodiments, the subject is administered the pharmaceutical composition as described herein at least twice a day. In certain embodiments, the subject is administered the pharmaceutical composition as described herein at least three times a day. In other embodiments, the subject is administered the pharmaceutical composition up to once a day. In other embodiments, the subject is administered the pharmaceutical composition up to twice a day. In other embodiments, the subject is administered the pharmaceutical composition up to three times a day. In certain embodiments, the subject is administered the pharmaceutical composition not more than once a day. In certain embodiments, the subject is administered the pharmaceutical composition not more than twice a day. In certain embodiments, the subject is administered the pharmaceutical composition not more than three times a day. In certain embodiments, the subject is administered the pharmaceutical composition as needed. In certain embodiments, the subject is administered the pharmaceutical composition as needed, but not more than once a day. In certain embodiments, the subject is administered the pharmaceutical composition as needed, but not more than twice a day. In certain embodiments, the subject is administered the pharmaceutical composition as needed, but not more than three times a day. In certain embodiments, the subject is administered the pharmaceutical composition at least once every other day. In certain embodiments, the subject is administered the pharmaceutical composition as described herein at least twice every other day. In certain embodiments, the subject is administered the pharmaceutical composition as described herein at least three times every other day.

In some embodiments of the foregoing methods, the pharmaceutical composition described herein may be administered for at least about 3 days to about 30 days, at least about 4 days to about 7 days, up to about 10 days, at least about 2 weeks, at least about 4 weeks, at least about 8 weeks, at least about 14 weeks, at least about 16 weeks, at least about 24 weeks, at least about 1 year, at least about 2 years, or the duration of the subject's life. In some embodiments, the pharmaceutical composition may be administered for at least 3, 4, 5, 6, 7, 8, 9, 10, 11, 12, 13, 14, 15, 16, 17, 18, 19, 20, 21, 22, 23, 24, 25, 26, 27, 28, 29, or 30 days. In some embodiments, the pharmaceutical composition may be administered for at most 3, 4, 5, 6, 7, 8, 9, 10, 11, 12, 13, 14, 15, 16, 17, 18, 19, 20, 21, 22, 23, 24, 25, 26, 27, 28, 29, or 30 days. In some embodiments, the pharmaceutical composition may be administered for about 3, 4, 5, 6, 7, 8, 9, 10, 11, 12, 13, 14, 15, 16, 17, 18, 19, 20, 21, 22, 23, 24, 25, 26, 27, 28, 29, or 30 days.

In some embodiments of the foregoing methods, where the subject has an acute condition, the pharmaceutical composition is administered QD or QID for at least 4, 5, 6, 7, 8, 9, or 10 days, or at most 4, 5, 6, 7, 8, 9, or 10 days, or about 4, 5, 6, 7, 8, 9, or 10 days. In some embodiments of the foregoing methods, where the subject has an acute condition, the pharmaceutical composition is administered QD or QID for about 4 days to about 10 days.

In some embodiments of the foregoing methods, where the subject has a chronic condition, the pharmaceutical composition is administered QD or every other day for at least 14, 15, 16, 17, 18, 19, 20, 22, 23, 24, 25, 26, 27, or 28 days; or at most 14, 15, 16, 17, 18, 19, 20, 22, 23, 24, 25, 26, 27, or 28 days; or about 14, 15, 16, 17, 18, 19, 20, 22, 23, 24, 25, 26, 27, or 28 days. In some embodiments of the foregoing methods, where the subject has a chronic condition, the pharmaceutical composition is administered QD or every other day for about 2 weeks to about 4 weeks.

In some embodiments of the foregoing methods, a reduction in a subject's mortality risk may be described as an increase or extension of a subject's life span by at least 30 days, 60 days, 90 days, 120 days, 180 days, 360 days, 1 year, or 2 years as compared to an average life span for similarly situated subjects treated with opioid therapy, of similar age, and having the same or similar underlying disease or condition.

In some embodiments of the foregoing methods, the subject may be suffering from, and/or has previously suffered from, an opioid-induced bowel disorder, such as opioid-induced bowel dysfunction (OIBD) or opioid-induced constipation. In some embodiments, the subject may have opioid-induced bowel dysfunction (OIBD), wherein OIBD is defined as <3 spontaneous bowel movements per week and hard or lumpy stools and/or a sensation of incomplete evacuation and/or straining in ≥25% of bowel movements. In some embodiments, the subject does not have, and/or has not previously suffered from, opioid-induced bowel dysfunction (OIBD). In some embodiments, the subject previously suffered from opioid-induced bowel dysfunction (OIBD).

In some embodiments, the subject may have, and/or has previously suffered from, opioid-induced bowel constipation (OIC), wherein the subject has OIC for at least one day, about 1 hour to about 30 days, or at least 30 days, and wherein the subject has experienced less than 3 rescue free bowel movements for at least one week or less than 3 rescue free bowel movements per week for at least four consecutive weeks. In some embodiments, the subject does not have, and/or has not previously suffered from, opioid induced constipation (OIC). In some embodiments, the subject previously suffered from opioid induced constipation (OIC).

In some embodiments of the foregoing methods, the method comprises determining whether a subject has, and/or has previously had, an increased risk of death based on the presence of various risk factors, for example, as listed under Table 3. In some embodiments of the foregoing methods, the method comprises determining whether a subject is a woman, is less than 60 years of age, has cancer, and/or has a chronic condition (i.e., an advanced illness, a cancer, and/or a chronic non-cancer pain).

The invention also provides kits for providing one or more of the foregoing methods. The kits include a pharmaceutical composition described herein or a combination of pharmaceutical compositions described herein either alone or in combination in suitable packaging, and written material that can include instructions for use, discussion of clinical studies, and listing of side effects.

The kits described herein are for use in reducing or ameliorating mortality risk in a subject as described herein by administering one or more pharmaceutical compositions that include MNTX as an active ingredient.

In order that the invention described herein may be more fully understood, the following examples are set forth. It should be understood that these examples are for illustrative purposes only and are not to be construed as limiting this invention in any manner.

All features of each of the aspects of the invention apply to all other aspects mutatis mutandis. The contents of all references, patents, pending patent applications and published patents, cited throughout this application are hereby expressly incorporated by reference.

### ENUMERATED EMBODIMENTS

Embodiment 1. A composition comprising methylnaltrexone (MNTX), or a salt thereof, for use in reducing mortality risk in a subject suffering from an underlying disease or condition, wherein the composition is preferably administered to the subject in an effective amount.

Embodiment 2. The composition for use according to embodiment 1, wherein the underlying disease or condition is selected from the group consisting of an advanced illness, cancer, a chronic non-cancer pain, postoperative ileus, and recovery from orthopedic surgery.

Embodiment 3. The composition for use according to embodiment 2, wherein the subject is suffering from an advanced illness.

Embodiment 4. The composition for use according to embodiment 2 or 3, wherein the subject is suffering from a cancer.

Embodiment 5. The composition for the use according to embodiments 1-3, wherein the subject is not suffering from cancer.

Embodiment 6. The composition for use according to embodiment 2, wherein the subject is suffering from a chronic non-cancer pain.

Embodiment 7. The composition for use according to embodiment 2, wherein the subject is suffering from postoperative ileus.

Embodiment 8. The composition for use according to embodiment 2, wherein the subject has undergone orthopedic surgery.

Embodiment 9. The composition for use according to any one of the preceding embodiments, wherein the subject is female.

Embodiment 10. The composition for use according to any one of the preceding embodiments, wherein the subject is less than 60, less than 50, less than 40 or less than 30 years of age.

Embodiment 11. The composition for use according to embodiment 4, wherein the cancer is selected from the group consisting of a carcinoma, a sarcoma, a melanoma, a lymphoma, a leukemia, and a blastoma.

Embodiment 12. The composition for use according to embodiment 4 or 11, wherein the cancer is selected from the group consisting of a breast cancer, a liver cancer, a head and neck cancer, an esophageal cancer, a stomach cancer, a small intestine cancer, a colon cancer, a rectal cancer, an anal cancer, a skin cancer, a glandular cancer, a circulatory cancer, a prostate cancer, a pancreatic cancer, a hematopoietic cancer, a bone marrow cancer, a bone cancer, a cartilage cancer, a fat cancer, a brain cancer, a nerve cancer, a lung cancer, and a lymph cancer.

Embodiment 13. The composition for use according to any one of the preceding embodiments, wherein the subject has a chronic condition.

Embodiment 14. The composition for use according to embodiment 13, wherein the chronic condition is selected from the group consisting of an advanced illness, a cancer, and a chronic non-cancer pain.

Embodiment 15. The composition for use according to any one of the preceding embodiments, wherein the subject has an acute condition.

Embodiment 16. The composition for use according to embodiment 15, wherein the acute condition is postoperative ileus.

Embodiment 17. The composition for use according to any one of the preceding embodiments, wherein the subject has a cardiovascular risk factor.

Embodiment 18. The composition for use according to embodiment 17, wherein the cardiovascular risk factor is selected from the group consisting of hyperlipidemia/hypercholesterolemia, diabetes mellitus, stroke, myocardial infarction, hypertension, and angina.

Embodiment 19. The composition for use according to any one of the preceding embodiments, wherein the composition comprises one or more of a tablet, a capsule, a sachet, a liquid solution, a powder for suspension, or a packaged composition, optionally, wherein the composition is administered orally, intravenously, or subcutaneously.

Embodiment 20. The composition for use according to any one of the preceding embodiments, wherein the composition is administered at a dosage of between about 0.001 to about 100 mg/kg body weight or between about 0.1 mg to about 1500 mg of MNTX, or a salt thereof.

Embodiment 21. The composition for use according to any one of the preceding embodiments, wherein the composition is administered orally, optionally, at a dosage of about 10 mg, about 50 mg, about 150 mg, about 300 mg, about 450 mg, or about 600 mg of MNTX, or a salt thereof, optionally, wherein the composition is administered as one or more tablets, optionally, wherein the tablet comprises about 150 mg of MNTX, or a salt thereof.

Embodiment 22. The composition for use according to any one of the preceding embodiments, wherein the composition is administered subcutaneously, optionally, at a dosage of about 8 mg, about 12 mg, or about 0.15 mg/kg bodyweight or about 0.30 mg/kg bodyweight of MNTX, or a salt thereof.

Embodiment 23. The composition for use according to any one of the preceding embodiments, wherein the composition is administered intravenously, optionally, at a dosage of about 12 mg or about 24 mg of MNTX, or a salt thereof.

Embodiment 24. The composition for use according to any one of the preceding embodiments, wherein the composition is administered at least every 6 hours, at least every 12 hours, at least once a day (e.g., as a single dose), or at least once every other day, optionally, wherein the composition is administered at least once a day (QD), at least twice a day (BID), at least three times a day (TID), or continuously.

Embodiment 25. The composition for use according to embodiments 19-23, wherein the dosage is a daily dosage.

Embodiment 26. The composition for use according to any one of the preceding embodiments, wherein the subject is administered the composition for at least about 3 days to about 30 days, at least about 4 days to about 7 days, up to about 10 days, at least about 2 weeks, at least about 4 weeks, at least about 8 weeks, at least about 14 weeks, at least about 16 weeks, at least about 24 weeks, at least about 1 year, at least about 2 years, or the duration of the subject's life, optionally, wherein the subject is administered the composition for the duration of their treatment for an underlying condition, optionally, wherein the underlying condition is an advanced illness, optionally, wherein the advanced illness is a cancer.

Embodiment 27. The composition for use according to any one of the preceding embodiments, wherein the subject has received, or is receiving, an opioid treatment comprising the administration of at least one opioid, optionally, wherein the opioid treatment is administered orally, transdermally, intravenously, or subcutaneously.

Embodiment 28. The composition for use according to embodiments 26, wherein the subject has received opioid treatment comprising from about 10 mg to about 500 mg, about 20 mg to about 300 mg, or about 25 mg to about 200 mg of oral morphine equivalents per day.

Embodiment 29. The composition for use according to embodiments 26 or 27, wherein the subject has received opioid treatment for at least 1 day, at least 7 days, at least 14 days, or at least 30 days, or wherein the subject will start opioid treatment in less than about 1 week, about 2 weeks, about 3 weeks, or about 4 weeks, optionally, wherein the subject has received opioid treatment comprising at least about 50 mg of oral morphine equivalents per day for at least about 14 days.

Embodiment 30. The composition for use according to any one of the preceding embodiments, wherein the subject is suffering from an opioid-induced bowel disorder (e.g., opioid-induced bowel dysfunction (OIBD) or opioid-induced constipation (OIC)), optionally, wherein:

(i) the subject has opioid-induced bowel dysfunction (OIBD), optionally, wherein OIBD is defined as <3 spontaneous bowel movements per week and hard or lumpy stools and/or a sensation of incomplete evacuation and/or straining in ≥25% of bowel movements, or

(ii) the subject has opioid-induced bowel constipation (OIC), optionally, wherein the subject has OIC for at least one day, about 1 hour to about 30 days, or at least 30 days, optionally, wherein the subject has experienced less than 3 rescue free bowel movements for at least one week or less than 3 rescue free bowel movements per week for at least four consecutive weeks.

Embodiment 31. The composition for use according to any one of the preceding embodiments, further comprising administering an additional therapy to the subject, optionally, wherein the additional therapy comprises an anticancer agent, optionally, wherein the anticancer agent is selected from the group consisting of a chemotherapeutic agent, an immunotherapy agent, a radiotherapy, and an anti-angiogenic agent, optionally, wherein the additional therapy comprises surgery.

Embodiment 32. The composition for use according to any one of the preceding embodiments, wherein the composition comprises methylnaltrexone bromide.

Embodiment 33. The composition for use according to any one of the preceding embodiments, wherein the composition extends the subject's life span by at least 30, 60, 90, 120, 180 or 360 days.

Embodiment 34. A methylnaltrexone pharmaceutical composition for use in reducing mortality risk in a subject receiving opioid therapy, wherein the methylnaltrexone pharmaceutical composition is selected from the group consisting of a methylnaltrexone pharmaceutical composition for oral administration, a methylnaltrexone ion pair pharmaceutical composition, and a methylnaltrexone pharmaceutical composition for subcutaneous administration.

Embodiment 35. The methylnaltrexone pharmaceutical composition for use according to embodiment 34, wherein the subject has an underlying disease or condition.

Embodiment 36. The methylnaltrexone pharmaceutical composition for use according to embodiment 35, wherein the underlying disease or condition is selected from the group consisting of an advanced illness, cancer, a chronic non-cancer pain, postoperative ileus, and recovery from orthopedic surgery, and combinations thereof.

Embodiment 37. The methylnaltrexone pharmaceutical composition for use according to any one of embodiments 34 to 36, wherein the subject receiving opioid therapy has cancer.

Embodiment 38. The methylnaltrexone pharmaceutical composition for use according to any one of embodiments 34 to 37, wherein the subject receiving opioid therapy is less than 60 years of age.

Embodiment 39. The methylnaltrexone pharmaceutical composition for use according to any one of embodiments 34 to 38, wherein the subject receiving opioid therapy has a chronic condition.

Embodiment 40. The methylnaltrexone pharmaceutical composition for use according to embodiment 39, wherein the chronic condition is selected from the group consisting of an advanced illness, cancer, a chronic non-cancer pain, and combinations thereof.

Embodiment 41. The methylnaltrexone pharmaceutical composition for use according to embodiments 34 to 40, wherein the opioid therapy comprises administration of an opioid to the subject.

Embodiment 42. The methylnaltrexone pharmaceutical composition for use according to embodiment 41, wherein the opioid comprises an opioid analgesic.

Embodiment 43. The methylnaltrexone pharmaceutical composition for use according to embodiment 41, wherein the opioid is selected from the group consisting of alfentanil, anileridine, asimadoline, bremazocine, burprenorphine, butorphanol, codeine, dezocine, diacetylmorphine (heroin), dihydrocodeine, diphenoxylate, ethylmorphine, fedotozine, fentanyl, funaltrexamine, hydrocodone, hydromorphone, levallorphan, levomethadyl acetate, levorphanol, loperamide, meperidine (pethidine), methadone, morphine, morphine glucoronide, nalbuphine, nalorphine, nicomorphine, opium, oxycodone, oxymorphone, papaveretum, pentazocine, propiram, propoxyphene, remifentanyl, sufentanil, tilidine, trimebutine, and tramadol, and a combination thereof.

Embodiment 44. The methylnaltrexone pharmaceutical composition for use according to any one of embodiments 34 to 40, wherein the use comprises administering an opioid to the subject.

Embodiment 45. The methylnaltrexone pharmaceutical composition for use according to embodiment 44, wherein the opioid comprises an opioid analgesic.

Embodiment 46. The methylnaltrexone pharmaceutical composition for use according to embodiment 44, wherein the opioid is selected from the group consisting of alfentanil, anileridine, asimadoline, bremazocine, burprenorphine, butorphanol, codeine, dezocine, diacetylmorphine (heroin), dihydrocodeine, diphenoxylate, ethylmorphine, fedotozine, fentanyl, funaltrexamine, hydrocodone, hydromorphone, levallorphan, levomethadyl acetate, levorphanol, loperamide, meperidine (pethidine), methadone, morphine, morphine-6-glucoronide, nalbuphine, nalorphine, nicomorphine, opium, oxycodone, oxymorphone, papaveretum, pentazocine, propiram, propoxyphene, remifentanyl, sufentanil, tilidine, trimebutine, and tramadol, and a combination thereof.

Embodiment 47. The methylnaltrexone pharmaceutical composition for use according to any one of embodiments 34 to 46, wherein a methylnaltrexone pharmaceutical composition for oral administration is administered to the subject.

Embodiment 48. The methylnaltrexone pharmaceutical composition for use according to any one of embodiments 34 to 47, wherein a methylnaltrexone ion pair pharmaceutical composition is administered to the subject.

Embodiment 49. The methylnaltrexone pharmaceutical composition for use according to any one of embodiments 34 to 48, wherein a methylnaltrexone pharmaceutical composition for subcutaneous administration is administered to the subject.

Embodiment 50. The methylnaltrexone pharmaceutical composition for use according to any one of embodiments 34 to 49, wherein the methylnaltrexone pharmaceutical composition is administered intermittently, QD, BID, TID, or QID.

Embodiment 51. The methylnaltrexone pharmaceutical composition for use according to any one of embodiments 34 to 49, wherein the methylnaltrexone pharmaceutical composition is administered at least every 6 hours.

Embodiment 52. The methylnaltrexone pharmaceutical composition for use according to any one of embodiments 34 to 49, wherein the methylnaltrexone pharmaceutical composition is administered at least every 12 hours.

Embodiment 53. The methylnaltrexone pharmaceutical composition for use according to any one of embodiments 34 to 49, wherein the methylnaltrexone pharmaceutical composition is administered at least once every other day.

Embodiment 54. The methylnaltrexone pharmaceutical composition for use according to any one of embodiments 1 to 53, wherein the subject is suffering from an opioid-induced bowel disorder.

Embodiment 55. The methylnaltrexone pharmaceutical composition for use according to embodiment 54, wherein the opioid-induced bowel disorder is opioid-induced bowel dysfunction (OIBD) or opioid-induced constipation (OIC).

Embodiment 56. The methylnaltrexone pharmaceutical composition for use according to any one of embodiments 1 to 53, wherein the subject is not suffering from an opioid-induced bowel disorder.

Embodiment 57. The methylnaltrexone pharmaceutical composition for use according to embodiment 56, wherein the opioid-induced bowel disorder is opioid-induced bowel dysfunction (OIBD) or opioid-induced constipation (OIC).

Embodiment 58. The methylnaltrexone pharmaceutical composition for use according to any one of embodiments 1-57, further comprising determining whether a subject has, and/or has previously had, an increased risk of death based on the presence of various risk factors, optionally, as listed under Table 3.

### EXAMPLES

The following Examples are merely illustrative and are not intended to limit the scope or content of the disclosure in any way.

### Example 1—Reductions in all-Cause Mortality Associated with the Use of Methylnaltrexone for Opioid-Induced Bowel Disorders: A Pooled Analysis

Clinical trial data was analyzed to determine the effect of MNTX therapy on opioid related all-cause mortality risk. The study population included patients with noncancer pain or advanced illness who received subcutaneous, intravenous, or oral MNTX or matching placebo in 12 phase 2 to 4, randomized, double-blind, placebo-controlled studies. The characteristics of the studies (duration, dosage, patient disposition, development phase) are summarized in Table 1. Study protocols were approved by an investigational review board or an independent ethics committee for each individual study. Written informed consent was obtained from patients before entering each study. Adherence to Good Clinical Practice and the Declaration of Helsinki were practiced. Each clinical trial was registered in clinicaltrials.gov (NCT00387309 [MNTX 300], NCT00401375 [MNTX 3301], NCT00672477 [MNTX 4000], NCT00547586 [MOA-728-2201], NCT00605644 [MOA-728-2202], NCT00402038 [MNTX 302], NCT00401362 [MNTX 301], NCT01186770 [MNTX 3201], NCT00529087 [MNTX 3356], NCT00640146 [MNTX 2101], NCT00366431 [MOA-728 200], NCT00528970 [MOA-728 301].

TABLE 1
Randomized, double-blind, placebo-controlled studies
of methylnaltrexone included in pooled analysis.

| | | Patient | Group Ns* | | |
|---|---|---|---|---|---|
| Study | Phase | Population | MNTX | PBO | MNTX Dosage |
| MNTX 2101 [2] | 2 | Acute OIC after | 18 | 15 | 12 mg SC MNTX once |
| | | orthopedic surgery | | | daily for 4-7 days |
| MOA-728 0200 | 2 | OIBD and chronic | 192 | 44 | 10, 50, 150, 300, or 450 mg |
| | | noncancer pain | | | oral MNTX once daily for 4 weeks |
| MOA-728 2201 | 2 | OIBD and chronic | 89 | 33 | 150, 300, 450, or 600 mg |
| | | noncancer pain | | | oral MNTX once daily for 4 weeks |
| MOA-728 2202 | 2 | OIBD and chronic | 99 | 29 | 150, 300, 450, or 600 mg |
| | | noncancer pain | | | oral MNTX once daily for 4 weeks |
| MOA-728 300 [38]^{†} | 3 | Postoperative | 357 | 176 | 12 or 24 mg IV MNTX every 6 hours |
| | | ileus | | | for up to 10 days |
| MNTX 3301 [38]^{†} | 3 | Postoperative | 344 | 171 | 12 or 24 mg IV MNTX every 6 hours |
| | | ileus | | | for up to 10 days |
| MOA-728 301 | 3 | Postoperative | 249 | 124 | 12 or 24 mg IV MNTX every 6 hours |
| | | ileus | | | for up to 10 days |
| MNTX 301 [32] | 3 | OIC and | 102 | 52 | Single dose 0.15 or 0.30 mg/kg SC |
| | | advanced illness | | | MNTX |
| MNTX 302 [34] | 3 | OIC and | 62 | 71 | 0.15 mg/kg SC MNTX every other |
| | | advanced illness | | | day for 2 weeks (optional increase |
| | | | | | to 0.30 mg/kg for week 2) |
| MNTX 3356 [24] | 3 | OIC and chronic | 298 | 162 | 12 mg SC MNTX once daily or |
| | | noncancer pain | | | every other day for 4 weeks |
| MNTX 3201 [28] | 3 | OIC and chronic | 602 | 201 | 150, 300, or 450 mg oral MNTX |
| | | noncancer pain | | | once daily for 12 weeks |
| MNTX 4000 | 4 | OIC and | 116 | 114 | 8 or 12 mg SC MNTX every other |
| | | advanced illness | | | day for 14 days |

*Intent-to-treat population.
^{†}Studies MOA-728 300 and MNTX 3301 were phase 3 studies of identical design, described in a single publication [38].
IV, intravenous; MNTX, methylnaltrexone; OIBD, opioid-induced bowel dysfunction (defined as <3 spontaneous bowel movements per week and hard or lumpy stools, a sensation of incomplete evacuation and/or straining, in ≥25% of bowel movements); OIC, opioid-induced constipation; PBO, placebo; SC, subcutaneous.

### Assessments and Analyses

Demographics and baseline characteristics were pooled and described descriptively for age, gender, body mass index (BMI), and daily opioid consumption based on oral morphine equivalents (OME) for the pooled overall population and for the cohort of patients who died (mortality cohort). The presence of cardiovascular risk factors was also collected. All-cause mortality was defined as the number of patients who died ≤30 days after the final dose of study medication during the double-blind phase of each study.

To calculate the person-years of exposure (PYE) for those who died, the sum of exposure days before death was divided by 365.25 and then multiplied by 100. To calculate the PYE for those who survived, the sum of exposure days before last study visit was divided by 365.25 and then multiplied by 100. The mortality rate per 100 PYE was calculated as (number of deaths/PYE)×100. It should be noted that each study was of different duration ranging from 1 day to 12 weeks. Therefore, the duration of mortality follow-up ranged from approximately 1 to 4 months. Mortality risk for each treatment group was calculated for the overall population and for subgroups stratified by cancer vs noncancer, age <60 years vs ≥60 years, men vs women, and acute vs chronic diagnosis.

Mortality risks (P-values, hazard ratios [HR] and 95% confidence intervals [CI]) for patients receiving MNTX or placebo were compared using a Cox proportional hazards regression model, with only treatment effect in the model, not adjusting for other factors. The model was also analyzed with the following covariates: cancer status (cancer vs noncancer), age (<60 years vs ≥60 years) and gender (male vs female). P-values had a 2-sided nominal significance level of 0.05 without adjustment for multiplicity. The pattern of deaths over time among patients in the MNTX and placebo groups was also evaluated by Kaplan-Meier analysis.

### Results

This analysis included 2526 patients receiving MNTX and 1192 receiving placebo. Table 2 describes the disposition of patients in the overall population as well as in each of the stratified subgroups analyzed (cancer vs noncancer; age <60 or ≥60 years; men vs women; acute vs chronic diagnosis). Compared with the overall population, a greater proportion of patients in the mortality cohort had cancer, were at least 60 years old, and had a chronic diagnosis.

TABLE 2
Baseline disposition for the overall population and by subgroup.

| | Overall Population | | | | Mortality Cohort | | | |
|---|---|---|---|---|---|---|---|---|
| | MNTX | | PBO | | MNTX | | PBO | |
| Subgroups, n (%) | (n = 2526) | | (n = 1192) | | (n = 33) | | (n = 35) | |
| Cancer | 537 | (21.3) | 324 | (27.2) | 22 | (66.7) | 27 | (77.1) |
| Noncancer | 1989 | (78.7) | 868 | (72.8) | 11 | (33.3) | 8 | (22.9) |
| Age <60 years | 1631 | (64.6) | 701 | (58.8) | 6 | (18.2) | 11 | (31.4) |
| Age ≥60 years | 895 | (35.4) | 491 | (41.2) | 27 | (81.8) | 24 | (68.6) |
| Men | 1152 | (45.6) | 549 | (46.1) | 18 | (54.5) | 16 | (45.7) |
| Women | 1374 | (54.4) | 643 | (53.9) | 15 | (45.5) | 19 | (54.3) |
| Acute diagnosis | 966 | (38.2) | 486 | (40.8) | 9 | (27.3) | 4 | (11.4) |
| (postoperative ileus) | | | | | | | | |
| Chronic diagnosis | 1560 | (61.8) | 706 | (59.2) | 24 | (72.7) | 31 | (88.6) |
| (advanced illness | | | | | | | | |
| and chronic | | | | | | | | |
| noncancer pain) | | | | | | | | |

MNTX, methylnaltrexone; PBO, placebo.

Baseline demographics and cardiovascular risk factors for the overall and mortality populations are summarized in Table 3. Compared with the overall population, the mortality cohort had an older mean age, a greater proportion of patients with a BMI <30 kg/m², and a greater proportion of patients had hyperlipidemia/hypercholesterolemia, diabetes mellitus, myocardial infarction (MI), and stroke. In the overall population, patients receiving MNTX had a higher median OME dose at baseline (MNTX: 174.0 mg/d vs placebo: 134.8 mg/d), whereas the reverse was observed in the mortality cohort (MNTX: 125.0 mg/d vs placebo: 200.0 mg/d). However, it should be noted that the mortality cohort OME values were only available from studies MNTX 301, MNTX 302 and MNTX 4000, all of which included patients with advanced illness, including cancer. We also assessed the OME dose for the cancer only cohort and found that the median OME dose was similar in the MNTX group (192.5 mg/d) compared with the placebo group (200.0 mg/d). The median OME dose among cancer patients was higher than that observed in noncancer patients (MNTX=120.0 mg/d; placebo=80.0 mg/d).

TABLE 3
Demographics and baseline characteristics and cardiovascular
risk factors for the pooled analysis population.

| | Overall Population | | | | Mortality Cohort | | | |
|---|---|---|---|---|---|---|---|---|
| | MNTX | | PBO | | MNTX | | PBO | |
| Characteristic | (n = 2526) | | (n = 1192) | | n = 33 | | n = 35 | |
| Mean (range) age, years | 55.0 | | 57.1 | | 69.6 | | 66.2 | |
| | (18, 101) | | (19, 100) | | (27, 93) | | (39, 87) | |
| Body mass index, | | | | | | | | |
| kg/m², n (%) | | | | | | | | |
| <30 | 1552 | (61.4) | 769 | (64.5) | 28 | (84.8) | 28 | (80.0) |
| ≥30 | 967 | (38.3) | 418 | (35.1) | 5 | (15.2) | 7 | (20.0) |
| Missing | 7 | (0.3) | 5 | (0.4) | 0 | | 0 | |
| Cardiovascular risk | | | | | | | | |
| factors, n (%)*^{†} | | | | | | | | |
| Hypertension | 1094 | (43.3) | 509 | (42.7) | 6 | (18.2) | 2 | (5.7) |
| Hyperlipidemia/ | 1033 | (40.9) | 527 | (44.2) | 19 | (57.6) | 18 | (51.4) |
| hypercholesterolemia | | | | | | | | |
| Diabetes mellitus | 695 | (27.5) | 398 | (33.4) | 19 | (57.6) | 19 | (54.3) |
| Myocardial infarction | 380 | (15.0) | 247 | (20.7) | 12 | (36.4) | 15 | (42.9) |
| Stroke | 393 | (15.6) | 256 | (21.5) | 11 | (33.3) | 17 | (48.6) |
| Angina | 278 | (11.0) | 150 | (12.6) | 2 | (6.1) | 2 | (5.7) |

| | | | | |
|---|---|---|---|---|
| Daily opioid | 174.0 | 134.8 | 125.0 | 200.0 |
| consumption (OME), | | | | |
| mg, median (range)^{‡} | (4.5-33,120) | (8.0, 10,160) | (12.0, 4071) | (33.5, 10,160) |

*Some patients had multiple cardiovascular risk factors.
^{†}Risk factors affecting ≥ 10% of patients in either group.
^{‡}Calculated for studies MNTX301, MNTX302 and MNTX4000 only.
MNTX, methylnaltrexone; OME, oral morphine equivalents.

### Mortality Risk for the Overall Population

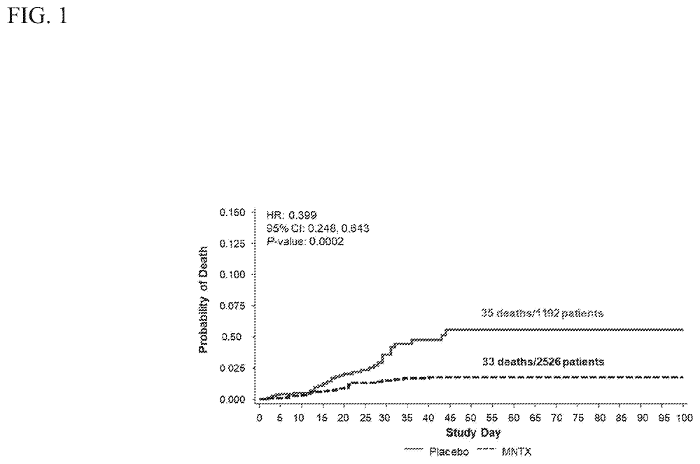

In the overall population, there were 33 deaths among 2526 patients who received MNTX and 35 deaths among 1192 patients who received placebo. This equated to a significant 60% reduction in all-cause mortality risk among patients treated with MNTX compared with placebo (HR: 0.399, 95% CI: 0.248, 0.643, P=0.0002) The mortality rate was 17.8 and 49.5 deaths/100 PYE for MNTX and placebo, respectively. A Kaplan-Meier analysis of study deaths over time is presented in FIG. 1. The curve temporally illustrates that deaths for patients receiving MNTX and placebo diverge early, with between-group differences emerging around day 15 before flattening due to the limited duration of studies and follow-up periods. Adjustments for cancer status, age group, and gender, resulted in a persistently significant attenuation of risk (HR: 0.508, 95% CI 0.314, 0.820, P=0.0056).

### Mortality Risk by Subgroup Stratification

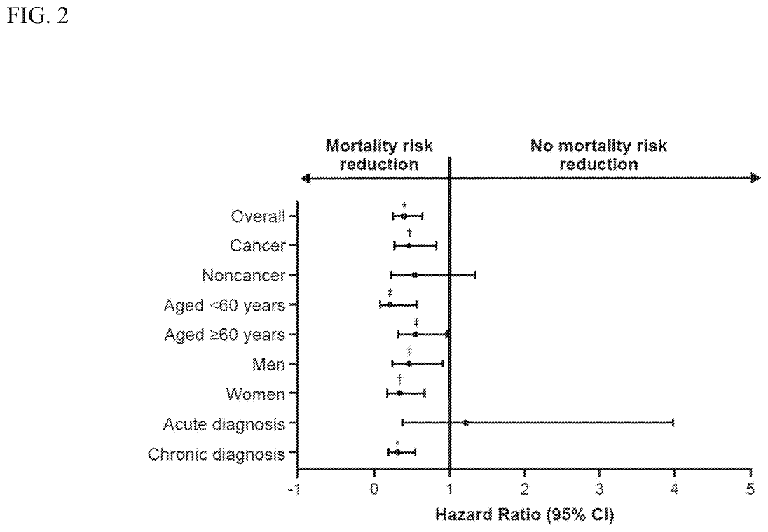

A mortality risk summary comparing HRs (95% CI) for the overall population and the stratified cohorts (cancer vs noncancer, age <60 years vs ≥60 years, men vs women, acute diagnosis vs chronic diagnosis) is presented in FIG. 2. In the stratified subgroups, each cohort except patients without cancer and those with an acute diagnosis had a significant mortality risk reduction when receiving MNTX.

Cancer vs Noncancer Diagnosis

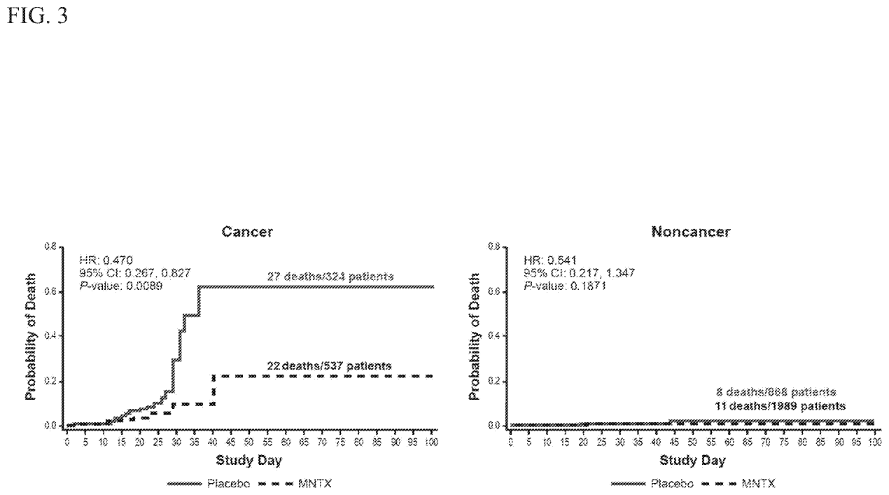

Patients with cancer receiving placebo had a significant increase in all-cause mortality compared with those who received MNTX (HR: 0.470, 95% CI: 0.267, 0.827, P=0.0089, FIG. 3). There was no difference between treatment groups in mortality risk among those without cancer (HR: 0.541, 95% CI: 0.217, 1.347, P=0.1871, FIG. 3).

Age <60 Years Vs ≥60 Years

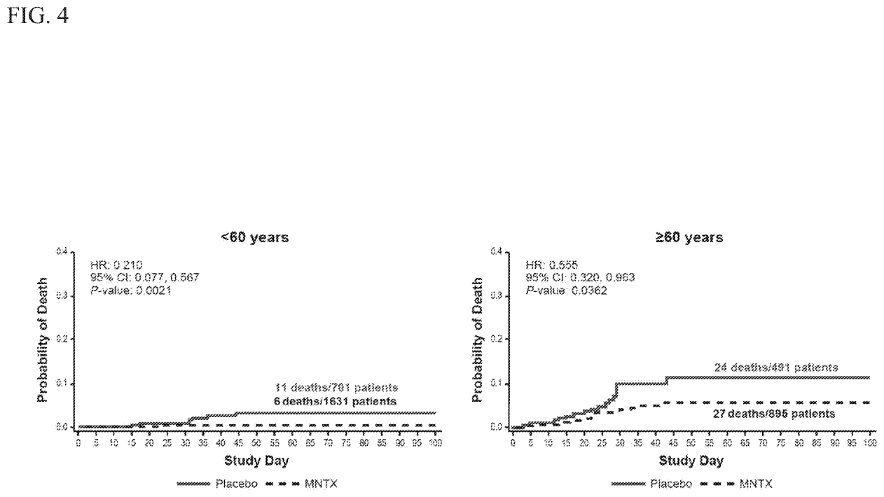

Younger patients (<60 years) who received MNTX had a 79% mortality risk reduction relative to those receiving placebo (HR: 0.210, 95% CI: 0.077, 0.567, P=0.0021, FIG. 4). For older patients (≥60 years), treatment with MNTX did not impact all-cause mortality to the same extent (HR: 0.555, 95% CI: 0.320, 0.963, P=0.0362, FIG. 4), but was still significant compared with placebo.

Women Vs Men

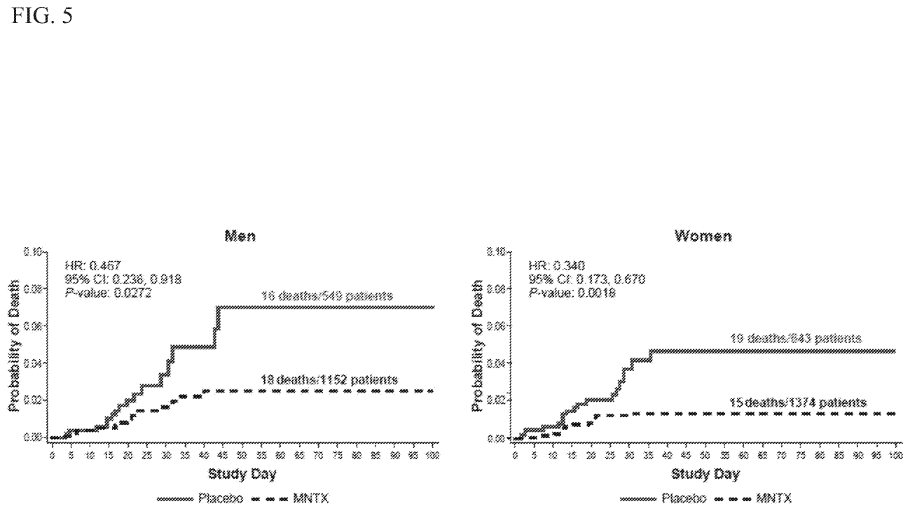

Women who received MNTX had a 66% mortality risk reduction compared with those receiving placebo (HR: 0.340, 95% CI: 0.173, 0.670, P=0.0018, FIG. 5). This effect was not as pronounced among men, who had a 53% mortality risk reduction (HR: 0.467, 95% CI: 0.238, 0.918, P=0.0272, FIG. 5).

Acute Diagnosis vs Chronic Diagnosis

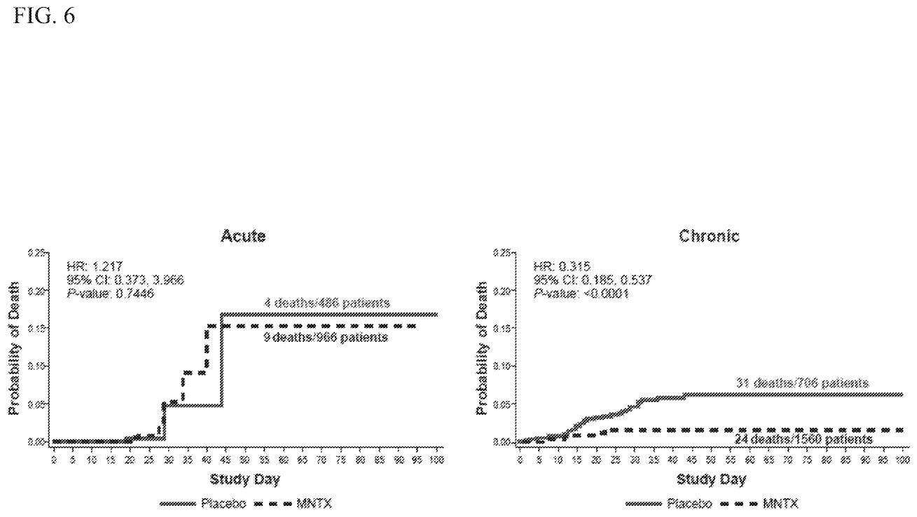

For patients with an acute diagnosis (postoperative ileus), there was no treatment effect on mortality risk (HR: 1.217, 95% CI: 0.373, 3.966, P=0.7446, FIG. 6). However, among those patients with a chronic diagnosis, which included patients with cancer and chronic noncancer pain, those receiving MNTX had a 68.5% reduction in mortality risk relative to those receiving placebo (HR: 0.315, 95% CI: 0.185, 0.537, P<0.0001, FIG. 6).

### Discussion

In this analysis of all-cause mortality in patients receiving MNTX for OIBD, mortality risk was reduced by 60% compared with patients receiving placebo. This reduction was substantial and statistically robust (P=0.0002). When stratification variables were applied, patients receiving MNTX who had cancer or a chronic diagnosis also had significant reductions in all-cause mortality. Both age and gender stratifications identified significant reductions in mortality risk with MNTX. These were more robust in younger patients and women.

These results support those of Janku and colleagues [16], who evaluated the effect of MNTX on OS in 2 placebo-controlled studies of opioid-treated patients with advanced illness and OIC. Patients with advanced cancer who received MNTX (n=117) had significantly longer median OS compared with those receiving placebo (n=112; 76 vs 56 days, respectively, P=0.033; HR: 0.63, 95% CI, 0.42, 0.95). Among cancer patients treated with MNTX, the 72 patients who responded (laxation within 4 hours for at least 2 of 4 first doses), compared with the 45 patients with nonresponse, had an even more substantial increase in OS (118 vs 58 days, respectively, P=0.001; HR: 0.37, 95% CI: 0.20, 0.67). The positive effects on OS in cancer patients was not observed among those with advanced illness.

Preclinical evidence has suggested that MOR agonism may promote tumor progression and metastasis, and that some of these effects may be reduced by a MOR antagonist like MNTX. Evidence suggests that MOR is overexpressed in lung and prostate tumors, and that MOR overexpression is associated with reduced progression-free and overall survival [31]. Morphine and other MOR agonists have been found to promote angiogenesis, and in turn cell proliferation and migration, in human dermal endothelial and pulmonary cells. For example, at clinically relevant levels, the angiogenic activity of morphine was 70% of that of vascular endothelial growth factor [31]. MOR agonists may also increase vascular permeability, further promoting migration and metastasis. In contrast, MNTX and other MOR antagonists can increase vascular barrier protection. MOR agonism may also promote the epithelial-mesenchymal transition, which paves the way to tumor cell metastasis [31]. In lung cancer cells, this transition involves interaction between MOR and growth factor receptors, and can be attenuated by MNTX [18,31]. Although these collective preclinical findings are promising, the exact effects that MOR agonism may have on OS and cancer progression are unknown.

Clinical data also support the possible role of MOR agonism in cancer progression. The use of nonopioid-containing anesthesia/analgesia regimens has been shown to reduce the risk of recurrence or metastasis in breast cancer, and the risk of recurrence in prostate cancer, compared with opioid-based perisurgical analgesia [13,31,41]. In a large prospective cohort study of women with breast cancer, use of strongly immunosuppressive opioids, such as morphine, increased the rate of all-cause mortality 4-fold [10]. Increased MOR expression has been associated with worse outcomes in prostate cancer, including reduced time to progression, progression-free survival, and OS. Increased opioid usage is also associated with worse outcomes and appears to be an independent contributor to progression risk [41]. Similarly, among patients with non-small cell lung cancer, pain severity and increased opioid requirements were found to be independent risk factors for reduced survival [42].

Activation of the MOR receptor itself may impact mortality risk. In a retrospective cohort study of critically ill patients, chronic opioid use was associated with a significant 2.20-fold increase in 90-day mortality [26]. The mortality risk was strongest among patients with cancer, which showed a 3.67-fold increase in 90-day mortality among chronic opioid users [26]. Interestingly, the 90-day mortality odds increased with an increase in morphine equivalent doses. In our analysis, the mortality cohort who received placebo had a greater median OME than those who received MNTX (200 mg/d vs 125 mg/d). Among cancer patients, the OME dose was 192.5 mg/d for MNTX and 200.0 mg/d for placebo; however, in noncancer patients the median OME dose was much lower (MNTX=120.0 mg/d; placebo=80.0 mg/d). The impact of mortality on opioid use was also depicted in a large 5-year analysis in patients using opioids chronically where mortality was significantly increased relative to nonopioid users (HR: 1.45, 95% CI: 1.28, 1.63, P<0.0001). This effect was also evident when stratified by cancer and noncancer patients [25].

In noncancer disease states, MOR agonism may contribute to sepsis risk; sepsis rates have risen in tandem with the opioid abuse crisis [3]. Enteric glial cells, which possess MOR, are responsible for enhancing barrier function via their effects on intestinal epithelial cells. Morphine (and presumably other MOR agonists) disrupts this barrier enhancement, possibly via down-regulation of glial-derived neurotrophic factor or through the disruption of epithelial tight junctions via upregulation of toll-like receptors, potentially increasing the risk of sepsis [3,22]. Morphine is also involved with the suppression of natural killer cell activity, depression of antibody production, and inhibition of cell growth [11,37]. Opioid use may also drive increased sepsis severity. A retrospective study of hospitalized sepsis patients found that in-hospital opioid use was associated with a 6-fold greater risk of death compared with no opioid use (HR: 6.24, 95% CI, 4.41, 8.83, P<0.0001) [40]. MOR agonists alter the gut microbiome as evidenced in a murine model of sepsis, that showed that morphine increased mortality and promoted overgrowth of gram-positive organisms more likely to disseminate beyond the intestine. These organisms upregulated the cytokine IL-17A, which induced inflammation and increased intestinal permeability, further facilitating bacterial dissemination [20]. A study of hospitalized patients with sepsis found that those treated with opioids compared with those not treated with opioids had significantly higher rates of gram-positive (39.3% vs 20.4%, P<0.0001), gram-negative (31.3% vs 27.0%, P=0.0019), and fungal infections (11.9% vs 2.2%, P<0.0001) [40]. Opioid immunosuppression has also been studied in human immunodeficiency virus (HIV). Meng and colleagues reviewed opioid-induced mechanisms such as disruption of GI homeostasis and microbial translocation, which were found to accelerate HIV progression [21]. Conversely, in preclinical models, MNTX has been shown to reverse the enhancement of HIV infection of macrophages and block HIV replication [14]. These findings must be viewed with caution since not all potential confounders have been accounted for and it is unknown if it is the pain that necessitates opioid use or the opioid use itself that contributes to the increased risk in mortality [19].

It is also possible that the constipation relief provided by MNTX may itself be contributing to reduced mortality. A Japanese questionnaire-based study found that, in comparison with daily (or more frequent) bowel movements (BMs), BM frequencies of 1 per 2 to 3 days and ≤1 per 4 days were associated with significantly increased cardiovascular disease mortality risk (multivariate-adjusted HR 1.21: 95% CI 1.08, 1.35; HR: 1.39, 95% CI: 1.06, 1.81; respectively) [15]. A quality of life outcomes study among patients in a supportive care setting found that constipation was a significant prognostic factor for mortality, although the effect size was small (HR: 1.02, P=0.0003) [6]. The role of persistent constipation vis-à-vis mortality risk remains unclear and deserves further study.

Notably it does not appear that the substantial reduction in mortality risk revealed in this analysis extends beyond MNTX to other PAMORAs. The PAMORA alvimopan, despite demonstrated efficacy in OIC treatment, was associated with a substantial increase in the risk for cardiovascular events compared with placebo (incidence rate for MI 1.30% vs 0%; for cardiovascular events 2.60% vs 1.12%, respectively). The observed elevation in cardiovascular risk led to restricting alvimopan to carefully controlled in-hospital use to prevent postoperative adverse events associated with opioids (ileus, delayed restoration of intestinal function) [5,12]. MNTX has not been associated with an increase in risk of cardiovascular events [29].

Limitations of the current analysis include its retrospective nature, its variability between studies with respect to study drug dose, duration and route of administration, and the inability to follow study subjects beyond prespecified time limits as these were short double-blind trials. In addition, the relatively small number of mortality events precluded subgroup analysis with respect to the cause of death and relationship to the disease state requiring opioid therapy. However, the strong signal to all-cause mortality reduction with MNTX treatment, especially given the similarity between MNTX and placebo groups in demographic and cardiovascular risk variables, suggests that the observed effect is real and has a basis in opioid/MOR biology and its effects on underlying disease states.

The results of this study demonstrate a statistically and clinically significant reduction in all-cause mortality among patients receiving MNTX for treatment of OIC compared with patients receiving placebo. This effect was consistent regardless of age and gender and observed patients with cancer or chronic diagnosis. We hypothesize that MNTX μ-receptor antagonism may provide protective benefit against the additional mortality risk associated with opioid treatment in patients with chronic diagnoses.

### REFERENCES

[1] Relistor [package insert]. Bridgewater, N.J.: Salix Pharmaceuticals, 2018.
[2] Anissian L, Schwartz H W, Vincent K, Vincent H K, Carpenito J, Stambler N, Ramakrishna T. Subcutaneous methylnaltrexone for treatment of acute opioid-induced constipation: phase 2 study in rehabilitation after orthopedic surgery. J Hosp Med 2012; 7(2):67-72.
[3] Bauman B D, Meng J, Zhang L, Louiselle A, Zheng E, Banerjee S, Roy S, Segura B J. Enteric glial-mediated enhancement of intestinal barrier integrity is compromised by morphine. J Surg Res 2017; 219:214-21.
[4] Bell T J, Panchal S J, Miaskowski C, Bolge S C, Milanova T, Williamson R. The prevalence, severity, and impact of opioid-induced bowel dysfunction: results of a US and European Patient Survey (PROBE 1). Pain Med 2009; 10(1):35-42.
[5] Brenner D M, Chey W D. An evidence-based review of novel and emerging therapies for constipation in patients taking opioid analgesics. Am J Gastroenterol Suppl 2014; 2(1):38-46.
[6] Brown J, Thorpe H, Napp V, Fairlamb D J, Gower N H, Milroy R, Parmar M K, Rudd R M, Spiro S G, Stephens R J, Waller D, West P, Peake M D. Assessment of quality of life in the supportive care setting of the big lung trial in non-small-cell lung cancer. J Clin Oncol 2005; 23(30):7417-27.
[7] Camilleri M, Drossman D A, Becker G, Webster L R, Davies A N, Mawe G M. Emerging treatments in neurogastroenterology: a multidisciplinary working group consensus statement on opioid-induced constipation. Neurogastroenterol Motil 2014; 26(10):1386-95.
[8] Carman W J, Su S, Cook S F, Wurzelmann J I, McAfee A. Coronary heart disease outcomes among chronic opioid and cyclooxygenase-2 users compared with a general population cohort. Pharmacoepidemiol Drug Saf 2011; 20(7):754-62.
[9] Coyne K S, Margolis M K, Yeomans K, King F R, Chavoshi S, Payne K A, LoCasale R J. Opioid-induced constipation among patients with chronic noncancer pain in the United States, Canada, Germany, and the United Kingdom: laxative use, response, and symptom burden over time. Pain Med 2015; 16(8):1551-65.
[10] Cronin-Fenton D P, Heide-Jørgensen U, Ahern T P, Lash T L, Christiansen P M, Ejlertsen B, Sjøgren P, Kehlet H, Sorensen H T. Opioids and breast cancer recurrence: A Danish population-based cohort study. Cancer 2015; 121(19):3507-14.
[11] Eisenstein T K. The Role of Opioid Receptors in Immune System Function. Front Immunol 2019; 10:2904.
[12] Entereg [package insert]. Whitehouse Station, N J: Merck Sharp & Dohme Corp, 2015.
[13] Exadaktylos A K, Buggy D J, Moriarty D C, Mascha E, Sessler D I. Can anesthetic technique for primary breast cancer surgery affect recurrence or metastasis? Anesthesiology 2006; 105(4): 660-4.
[14] Ho W Z, Guo C J, Yuan C S, Douglas S D, Moss J. Methylnaltrexone antagonizes opioid-mediated enhancement of HIV infection of human blood mononuclear phagocytes. J Pharmacol Exp Ther 2003; 307(3):1158-62.
[15] Honkura K, Tomata Y, Sugiyama K, Kaiho Y, Watanabe T, Zhang S, Sugawara Y, Tsuji I. Defecation frequency and cardiovascular disease mortality in Japan: The Ohsaki cohort study. Atherosclerosis 2016; 246:251-6.
[16] Janku F, Johnson L K, Karp D D, Atkins J T, Singleton P A, Moss J. Treatment with methylnaltrexone is associated with increased survival in patients with advanced cancer. Ann Oncol 2016; 27(11):2032-8.
[17] Kumar L, Barker C, Emmanuel A. Opioid-induced constipation: pathophysiology, clinical consequences, and management. Gastroenterol Res Pract 2014; 2014 doi: 10.1155/2014/141737:141737.
[18] Lennon F E, Mirzapoiazova T, Mambetsariev B, Poroyko V A, Salgia R, Moss J, Singleton P A. The mu opioid receptor promotes opioid and growth factor-induced proliferation, migration and epithelial mesenchymal transition (EMT) in human lung cancer. PLoS One 2014; 9(3):e91577.
[19] Macfarlane G J, Beasley M, Jones G T, Stannard C. The epidemiology of regular opioid use and its association with mortality: Prospective cohort study of 466 486 UK biobank participants. EClinicalMedicine 2020; 21:100321.
[20] Meng J, Banerjee S, Li D, Sindberg G M, Wang F, Ma J, Roy S. Opioid exacerbation of gram-positive sepsis, induced by gut microbial modulation, is rescued by IL-17A neutralization. Sci Rep 2015; 5:10918.
[21] Meng J, Sindberg G M, Roy S. Disruption of gut homeostasis by opioids accelerates HIV disease progression. Front Microbiol 2015; 6:643.
[22] Meng J, Yu H, Ma J, Wang J, Banerjee S, Charboneau R, Barke R A, Roy S. Morphine induces bacterial translocation in mice by compromising intestinal barrier function in a TLR-dependent manner. PLoS One 2013; 8(1):e54040.
[23] Meuser T, Pietruck C, Radbruch L, Stute P, Lehmann K A, Grond S. Symptoms during cancer pain treatment following WHO-guidelines: a longitudinal follow-up study of symptom prevalence, severity and etiology. Pain 2001; 93(3):247-57.
[24] Michna E, Blonsky E R, Schulman S, Tzanis E, Manley A, Zhang H, Iyer S, Randazzo B. Subcutaneous methylnaltrexone for treatment of opioid-induced constipation in patients with chronic, nonmalignant pain: a randomized controlled study. J Pain 2011; 12(5):554-62.
[25] Oh T K, Jeon Y T, Choi J W. Trends in chronic opioid use and association with five-year survival in South Korea: a population-based cohort study. Br J Anaesth 2019; 123(5):655-63.
[26] Oh T K, Song I A, Lee J H, Lim C, Jeon Y T, Bae H J, Jo Y H, Jee H J. Preadmission chronic opioid usage and its association with 90-day mortality in critically ill patients: a retrospective cohort study. Br J Anaesth 2019; 122(6):e189-e97.
[27] Panchal S J, Muller-Schwefe P, Wurzelmann J I. Opioid-induced bowel dysfunction: prevalence, pathophysiology and burden. Int J Clin Pract 2007; 61(7):1181-7.
[28] Rauck R, Slatkin N E, Stambler N, Harper J R, Israel R J. Randomized, double-blind trial of oral methylnaltrexone for the treatment of opioid-induced constipation in patients with chronic noncancer pain. Pain Pract 2017; 17(6):820-8.
[29] Rauck R L, Slatkin N E, Stambler N, Israel R J. Safety of oral methylnaltrexone for opioid-induced constipation in patients with chronic noncancer pain. J Pain Res 2019; 12:139-50.
[30] Ray W A, Chung C P, Murray K T, Hall K, Stein C M. Prescription of long-acting opioids and mortality in patients with chronic noncancer pain. JAMA 2016; 315(22):2415-23.
[31] Singleton P A, Moss J, Karp D D, Atkins J T, Janku F. The mu opioid receptor: a new target for cancer therapy? Cancer 2015; 121(16):2681-8.
[32] Slatkin N, Thomas J, Lipman A G, Wilson G, Boatwright M L, Wellman C, Zhukovsky D S, Stephenson R, Portenoy R, Stambler N, Israel R. Methylnaltrexone for treatment of opioid-induced constipation in advanced illness patients. J Support Oncol 2009; 7(1):39-46.
[33] Solomon D H, Rassen J A, Glynn R J, Lee J, Levin R, Schneeweiss S. The comparative safety of analgesics in older adults with arthritis. Arch Intern Med 2010; 170(22):1968-76.
[34] Thomas J, Karver S, Cooney G A, Chamberlain B H, Watt C K, Slatkin N E, Stambler N, Kremer A B, Israel R J. Methylnaltrexone for opioid-induced constipation in advanced illness. N Engl J Med 2008; 328(22):2332-43.
[35] Webster L R, Michna E, Khan A, Israel R J, Harper J R. Long-term safety and efficacy of subcutaneous methylnaltrexone in patients with opioid-induced constipation and chronic noncancer pain: a phase 3, open-label trial. Pain Med 2017; 18(8):1496-504.
[36] Weschules D J, Bain K T, Reifsnyder J, McMath J A, Kupperman D E, Gallagher R M, Hauck W W, Knowlton C H. Toward evidence-based prescribing at end of life: a comparative analysis of sustained-release morphine, oxycodone, and transdermal fentanyl, with pain, constipation, and caregiver interaction outcomes in hospice patients. Pain Med 2006; 7(4):320-9.
[37] Wiese A D, Griffin M R, Schaffner W, Stein C M, Greevy R A, Mitchel E F, Grijalva C G. Long-acting Opioid Use and the Risk of Serious Infections: A Retrospective Cohort Study. Clin Infect Dis 2019; 68(11): 1862-9.
[38] Yu C S, Chun H K, Stambler N, Carpenito J, Schulman S, Tzanis E, Randazzo B. Safety and efficacy of methylnaltrexone in shortening the duration of postoperative ileus following segmental colectomy: results of two randomized, placebo-controlled phase 3 trials. Dis Colon Rectum 2011; 54(5):570-8.
[39] Yuan C S, Foss J F, O'Connor M, Toledano A, Roizen M F, Moss J. Methylnaltrexone prevents morphine-induced delay in oral-cecal transit time without affecting analgesia: a double-blind randomized placebo-controlled trial. Clin Pharmacol Ther 1996; 59(4):469-75.
[40] Zhang R, Meng J, Lian Q, Chen X, Bauman B, Chu H, Segura B, Roy S. Prescription opioids are associated with higher mortality in patients diagnosed with sepsis: A retrospective cohort study using electronic health records. PLoS One 2018; 13(1):e0190362.
[41] Zylla D, Gourley B L, Vang D, Jackson S, Boatman S, Lindgren B, Kuskowski M A, Le C, Gupta K, Gupta P. Opioid requirement, opioid receptor expression, and clinical outcomes in patients with advanced prostate cancer. Cancer 2013; 119(23):4103-10.
[42] Zylla D, Kuskowski M A, Gupta K, Gupta P. Association of opioid requirement and cancer pain with survival in advanced non-small cell lung cancer. Br J Anaesth 2014; 113(Suppl 1):i109-i16.
[43] Zylla D, Steele G, Shapiro A, Richter S, Gupta P. Impact of opioid use on health care utilization and survival in patients with newly diagnosed stage IV malignancies. Support Care Cancer 2018; 26(7):2259-66.

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of reducing mortality risk in a subject suffering from an underlying disease or condition, comprising administering to the subject an effective amount of a composition comprising methylnaltrexone (MNTX), or a salt thereof.

2. The method of claim 1, wherein the underlying disease or condition is selected from the group consisting of an advanced illness, cancer, a chronic non-cancer pain, postoperative ileus, and recovery from orthopedic surgery.

3-8. (canceled)

9. The method of claim 1, wherein the subject is female and/or wherein the subject is less than 60, less than 50, less than 40 or less than 30 years of age.

10. (canceled)

11. The method of claim 2, wherein the subject is suffering from cancer and wherein
(i) the cancer is selected from the group consisting of a carcinoma, a sarcoma, a melanoma, a lymphoma, a leukemia, and a blastoma; and/or
(ii) the cancer is selected from the group consisting of a breast cancer, a liver cancer, a head and neck cancer, an esophageal cancer, a stomach cancer, a small intestine cancer, a colon cancer, a rectal cancer, an anal cancer, a skin cancer, a glandular cancer, a circulatory cancer, a prostate cancer, a pancreatic cancer, a hematopoietic cancer, a bone marrow cancer, a bone cancer, a cartilage cancer, a fat cancer, a brain cancer, a nerve cancer, a lung cancer, and a lymph cancer.

12. (canceled)

13. The method of claim 1, (i) wherein the subject has a chronic condition, optionally, selected from the group consisting of an advanced illness, a cancer, and a chronic non-cancer pain; and/or (ii) wherein the subject has an acute condition, optionally, wherein the acute condition is postoperative ileus.

14-16. (canceled)

17. The method of claim 1, wherein the subject has a cardiovascular risk factor, optionally, selected from the group consisting of hyperlipidemia/hypercholesterolemia, diabetes mellitus, stroke, myocardial infarction, hypertension, and angina.

18. (canceled)

19. The method of claim 1,
(i) wherein the composition comprises one or more of a tablet, a capsule, a sachet, a liquid solution, a powder for suspension, or a packaged composition, optionally, wherein the composition is administered orally, intravenously, or subcutaneously;
(ii) wherein the composition is administered at a dosage of between about 0.001 to about 100 mg/kg body weight or between about 0.1 mg to about 1500 mg of MNTX, or a salt thereof;
(iii) wherein the composition is administered orally, optionally, at a dosage of about 10 mg, about 50 mg, about 150 mg, about 300 mg, about 450 mg, or about 600 mg of MNTX, or a salt thereof, optionally, wherein the composition is administered as one or more tablets, optionally, wherein the tablet comprises about 150 mg of MNTX, or a salt thereof;
(iv) wherein the composition is administered subcutaneously, optionally, at a dosage of about 8 mg, about 12 mg, or about 0.15 mg/kg bodyweight or about 0.30 mg/kg bodyweight of MNTX, or a salt thereof;
(v) wherein the composition is administered intravenously, optionally, at a dosage of about 12 mg or about 24 mg of MNTX, or a salt thereof;
(vi) wherein the composition is administered at least every 6 hours, at least every 12 hours, at least once a day (e.g., as a single dose), or at least once every other day, optionally, wherein the composition is administered at least once a day (QD), at least twice a day (BID), at least three times a day (TID), or continuously;
(vii) wherein the dosage is a daily dosage; and/or
(viii) wherein the subject is administered the composition for at least about 3 days to about 30 days, at least about 4 days to about 7 days, up to about 10 days, at least about 2 weeks, at least about 4 weeks, at least about 8 weeks, at least about 14 weeks, at least about 16 weeks, at least about 24 weeks, at least about 1 year, at least about 2 years, or the duration of the subject's life, optionally, wherein the subject is administered the composition for the duration of their treatment for an underlying condition, optionally, wherein the underlying condition is an advanced illness, optionally, wherein the advanced illness is a cancer.

20-26. (canceled)

27. The method of claim 1, wherein the subject has received, or is receiving, an opioid treatment comprising the administration of at least one opioid, optionally, wherein the opioid treatment is administered orally, transdermally, intravenously, or subcutaneously;
optionally, wherein the subject has received opioid treatment comprising from about 10 mg to about 500 mg, about 20 mg to about 300 mg, or about 25 mg to about 200 mg of oral morphine equivalents per day,
optionally, wherein the subject has received opioid treatment for at least 1 day, at least 7 days, at least 14 days, or at least 30 days, or wherein the subject will start opioid treatment in less than about 1 week, about 2 weeks, about 3 weeks, or about 4 weeks, optionally, wherein the subject has received opioid treatment comprising at least about 50 mg of oral morphine equivalents per day for at least about 14 days.

28-29. (canceled)

30. The method of claim 1, wherein the subject is suffering from an opioid-induced bowel disorder (e.g., opioid-induced bowel dysfunction (OIBD) or opioid-induced constipation (OIC)), optionally, wherein:
(i) the subject has opioid-induced bowel dysfunction (OIBD), optionally, wherein OIBD is defined as <3 spontaneous bowel movements per week and hard or lumpy stools and/or a sensation of incomplete evacuation and/or straining in ≥25% of bowel movements, or
(ii) the subject has opioid-induced bowel constipation (OIC), optionally, wherein the subject has OIC for at least one day, about 1 hour to about 30 days, or at least 30 days, optionally, wherein the subject has experienced less than 3 rescue free bowel movements for at least one week or less than 3 rescue free bowel movements per week for at least four consecutive weeks.

31. The method of claim 1, further comprising administering an additional therapy to the subject, optionally, wherein the additional therapy comprises an anticancer agent, optionally, wherein the anticancer agent is selected from the group consisting of a chemotherapeutic agent, an immunotherapy agent, a radiotherapy, and an anti-angiogenic agent, optionally, wherein the additional therapy comprises surgery.

32. The method of claim 1, wherein the composition comprises methylnaltrexone bromide.

33. The method of claim 1, wherein the method extends the subject's life span by at least 30, 60, 90, 120, 180 or 360 days.

34. A method of reducing mortality risk in a subject receiving opioid therapy comprising administering to the subject one or more methylnaltrexone pharmaceutical compositions selected from the group consisting of a methylnaltrexone pharmaceutical composition for oral administration, a methylnaltrexone ion pair pharmaceutical composition, and a methylnaltrexone pharmaceutical composition for subcutaneous administration, optionally,
wherein the subject has an underlying disease or condition, optionally, selected from the group consisting of an advanced illness, cancer, a chronic non-cancer pain, postoperative ileus, and recovery from orthopedic surgery, and combinations thereof.

35-36. (canceled)

37. A method of reducing mortality risk in a subject with a cancer, less than 60 years of age and/or with a chronic condition and receiving opioid therapy comprising administering to the subject one or more methylnaltrexone pharmaceutical compositions selected from the group consisting of a methylnaltrexone pharmaceutical composition for oral administration, a methylnaltrexone ion pair pharmaceutical composition, and a methylnaltrexone pharmaceutical composition for subcutaneous administration.

38-39. (canceled)

40. The method of claim 37, wherein the subject is suffering from a chronic condition selected from the group consisting of an advanced illness, cancer, a chronic non-cancer pain, and combinations thereof.

41. The method of claim 34, wherein the opioid therapy comprises administration of an opioid to the subject and/or wherein the method comprises administering an opioid to the subject;
optionally, wherein the opioid comprises an opioid analgesic,
optionally, wherein the opioid is selected from the group consisting of alfentanil, anileridine, asimadoline, bremazocine, burprenorphine, butorphanol, codeine, dezocine, diacetylmorphine (heroin), dihydrocodeine, diphenoxylate, ethylmorphine, fedotozine, fentanyl, funaltrexamine, hydrocodone, hydromorphone, levallorphan, levomethadyl acetate, levorphanol, loperamide, meperidine (pethidine), methadone, morphine, morphine-6-glucoronide, nalbuphine, nalorphine, nicomorphine, opium, oxycodone, oxymorphone, papaveretum, pentazocine, propiram, propoxyphene, remifentanyl, sufentanil, tilidine, trimebutine, and tramadol, and a combination thereof.

42-46. (canceled)

47. The method of claim 34,
(i) wherein the method comprises administering to the subject a methylnaltrexone pharmaceutical composition for oral administration;
(ii) wherein the method comprises administering to the subject a methylnaltrexone ion pair pharmaceutical composition;
(iii) wherein the method comprises administering to the subject a methylnaltrexone pharmaceutical composition for subcutaneous administration;
(iii) wherein the methylnaltrexone pharmaceutical composition is administered intermittently, QD, BID, TID, or QID,
(iv) wherein the methylnaltrexone pharmaceutical composition is administered at least every 6 hours;
(v) wherein the methylnaltrexone pharmaceutical composition is administered at least every 12 hours; and/or
(vi) wherein the methylnaltrexone pharmaceutical composition is administered at least once every other day.

48-53. (canceled)

54. The method of claim 34, wherein the subject is suffering from an opioid-induced bowel disorder, optionally, wherein the opioid-induced bowel disorder is opioid-induced bowel dysfunction (OIBD) or opioid-induced constipation (OIC).

55. (canceled)

56. The method of claim 1, wherein the subject is not suffering from an opioid-induced bowel disorder, optionally, wherein the opioid-induced bowel disorder is opioid-induced bowel dysfunction (OIBD) or opioid-induced constipation (OIC).

57. (canceled)

58. The method of claim 1, wherein the method further comprises determining whether a subject has, and/or has previously had, an increased risk of death based on the presence of various risk factors, optionally, as listed under Table 3.
